(19) United States
(12) Patent Application Publication
(10) Pub. No.: US 2023/0390334 A1
(43) Pub. Date: Dec. 7, 2023

(54) METHOD OF TREATMENT OF CANCER OR TUMOUR

(71) Applicant: Adaptimmune Limited, Abingdon, Oxfordshire (GB)

(72) Inventor: Nicholas Jonathan Pumphrey, Abingdon, Oxfordshire (GB)

(21) Appl. No.: 17/819,480

(22) PCT Filed: Feb. 12, 2021
(86) PCT No.: PCT/GB2021/050341

§ 371 Date: Aug. 12, 2022

Related U.S. Application Data
(60) Provisional application No. 62/976,493, filed on Feb. 14, 2020.

(51) Int. Cl. A61K 35/17; A61K 38/17; A61K 45/06; A61P 35/00

(52) U.S. Cl. CPC A61K 35/17; A61K 38/1774; A61K 45/06; A61P 35/00

(57) ABSTRACT

The present invention relates to a method of treating, preventing or delaying the progression of cancer and/or tumour in a subject comprising administering to the subject a treatment regimen comprising an effective amount of modified immunoresponsive cells expressing or presenting a heterologous T-cell receptor (TCR) having the property of binding to Fetoprotein (AFP) or antigenic peptide thereof, in particular the treatment of Hepatocellular carcinoma (HCC).

### FIELD OF INVENTION

The present invention relates to a method of treating, preventing or delaying the progression of cancer and/or tumour in a subject comprising administering to the subject a treatment regimen comprising an effective amount of modified immunoresponsive cells expressing or presenting a heterologous T-cell receptor (TCR) having the property of binding to Alpha Fetoprotein (AFP) or antigenic peptide thereof, in particular the treatment of Hepatocellular carcinoma (HCC).

### BACKGROUND

Hepatocellular carcinoma (HCC) is the fifth most common form of cancer worldwide and the third most common cause of cancer-related deaths. The disease is often closely correlated with cirrhotic liver disease. Hepatocellular carcinoma has one of the lowest reported 5 year survival rates of all malignancies, global annual incidence is 1.2 million and is likely to increase coincident with Hepatitis B and C prevalence.

Surgical resection is currently considered the standard curative strategy however this carries a high risk of recurrence and an additional risk of hepatic decompensation in the patients with cirrhosis. Resection is limited as a treatment option for a small number of patients with single nodules, good liver function and no underlying cirrhosis (classified as Child-Pugh class A), resection is not often considered as an option in patients with multiple tumours. Alternative therapy such as Radiofrequency ablation (RFA) offers no discernable advantages as first-line treatment for small tumours. Transarterial chemoembolization (TACE) is currently considered a standard treatment for the patients with intermediate-stage HCC, patients with compensated liver function (Child B up to 8 points), with large single nodule (<5 cm) or multifocal HCC without evidence of vascular invasion or extra hepatic spread. This is an invasive therapy that blocks or slows down the blood supply to a tissue or organ. It can be used to block the flow of blood to a tumour in an attempt to cause the cancer cells to die. TACE has been reported to achieve a partial response in 15%-62% patients, and has been used in treatment of intermediate-stage HCC which includes a heterogeneous population of the patients with variable tumour burden and liver function, i.e. Child-Pugh class A or some B, most class B. Radioembolization or selective internal radiation therapy (SIRT) has also been used as an alternative therapeutic option for intermediate-stage HCC.

General chemotherapy and targeted systemic chemotherapies have been trialled for advanced HCC and intermediate HCC using small molecule inhibitors of multiple signalling pathways common to HCC pathogenesis including vascular endothelial growth factor (VEGF), epidermal growth factor, Ras mitogen-activated protein kinase (MARK), insulin-like growth factor receptor, hepatocyte growth factor/c-MET, PI3K/PTEN/Akt/mammalian target of rapamycin (mTOR) and Wnt/β-Catenin pathways. Relevant trial treatment compounds include: Sunitinib (a multi-kinase blocker targeting VEGFR and PDGFR), Brivanib a selective inhibitor of fibroblastic growth factor receptor and VEGFR, Everolimus an inhibitor of mTOR, Tivantinib a MET receptor tyrosine kinase inhibitor, Linifanib a multi-kinase inhibitor targeting VEGFR and PDGFR, and Sorafenib inhibitor of Ras/MAPK pathway and many cell surface tyrosine (VEGF receptors, platelet-derived growth factor receptor- (PDGFR-) β, RET, c-KIT and FMS-like tyrosine kinase-3). Immunotherapy using PD1 inhibitors has also been trialled for HCC. Additionally Regorafenib demonstrates dual targeted VEGFR2-TIE2 tyrosine kinase inhibition, Cabozantinib is an inhibitor of the tyrosine kinases c-Met and VEGFR2, and also inhibits AXL and RET.

It is desirable therefore to provide a therapy for tumour and/or cancer treatment, such as treatment of HCC, which is cancer specific, capable of treating intermediate or late stage cancer or single or multiple solid tumours, particularly where there has been failure or recurrence following primary therapy or surgery, preferably also where the therapy minimises or reduces toxicity or side effects for example risk of systemic toxicity of chemotherapeutic agents.

The present invention relates to and exemplifies the treatment of cancer and/or tumour in a subject comprising administering to the subject a treatment regimen comprising an effective amount of modified T-cells expressing or presenting a heterologous T-cell receptor (TCR) having the property of binding to AFP and in particular, specifically binding to FMNKFIYEI (SEQ ID No: 1).

AFP is expressed during foetal development and is the main component of foetal serum. During development the protein is produced at very high levels by the yolk sac and liver and is later repressed. AFP expression is frequently reactivated in hepatocellular carcinoma and high levels of the protein are used as a diagnostic marker for the disease. There are four known epitopes derived from AFP: AFP158 (residues 158-166 of SEQ ID NO:51), AFP137 (residues 137-145 of SEQ ID NO:51), AFP325 (residues 325-334 of SEQ ID NO:51), and AFP542 (residues 542-550 of SEQ ID NO:51). In particular, the HLA-A2 restricted AFP158 peptide FMNKFIYEI (SEQ ID No: 1) provides a suitable target for novel immunotherapeutic interventions; this peptide is naturally processed and has isolated from liver carcinoma lines.

### SUMMARY OF THE INVENTION

According to a first aspect of the present invention there is provided a method of treating, preventing or delaying the progression of cancer and/or tumour in a subject comprising administering to the subject a treatment regimen comprising an effective amount of modified immunoresponsive cells expressing or presenting a heterologous T-cell receptor (TCR) binding to alpha-fetoprotein or an AFP antigenic peptide thereof.

According to the invention the TCR may bind alpha-fetoprotein or an antigenic peptide thereof, for example Human alpha-fetoprotein or alpha-fetoprotein of SEQ ID NO: 51 or an antigenic peptide thereof. The TCR may bind to an antigenic peptide comprising;
(a) residues 158-166 of SEQ ID NO:51 (AFP158),
(b) residues 137-145 of SEQ ID NO:51 (AFP137),
(c) residues 325-334 of SEQ ID NO:51 (AFP325), (d) residues 542-550 of SEQ ID NO:51 (AFP542), or
(e) residues FMNKFIYEI (SEQ ID No: 1).

### Specific Binding TCR

According to the present invention the heterologous TCR and modified immunoresponsive cells comprising the heterologous T cell receptor (TCR) may bind to or bind with high affinity to and/or specifically and/or selectively bind a cancer and/or tumour antigen or peptide antigen thereof for example alpha-fetoprotein or peptide antigen thereof.

According to the invention the heterologous TCR may bind or specifically and/or selectively bind to alpha-fetoprotein or peptide antigen thereof associated with a cancerous condition, tumour and/or cancer and/or presented by tumour or cancer cell or tissue.

According to the invention the cancerous condition, tumour and/or cancer and/or tumour is an AFP expressing cancer and/or tumour or expresses a peptide antigen thereof.

Accordingly, a heterologous TCR for use in accordance with the invention is capable of specifically binding, and/or binding with high affinity, and/or selectively binding to AFP, a peptide antigen thereof or peptide antigen comprising the sequence FMNKFIYEI (SEQ ID No: 1), optionally in complex with a peptide presenting molecule for example major histocompatibility complex (MHC) or an HLA, optionally class I or II, for example with HLA-A2, or selected from HLA-A*02:01, HLA-A*02:02, HLA-A*02:03, HLA-A*02:04, HLA-A*02:05, HLA-A*02:06, HLA-A*02:642 or HLA-A*02:07, preferably HLA-A*02:01 or HLA-A*02:642.

Alternatively the heterologous T cell receptor (TCR), and modified immunoresponsive cells comprising the heterologous T cell receptor (TCR) may bind or specifically and/or selectively bind and/or bind with high affinity to an endogenously expressed tumour cell surface alpha-fetoprotein or peptide antigen thereof optionally wherein the binding is independent of presentation of the cell surface antigen as a complex with an peptide-presenting or antigen-presenting molecule, for example major histocompatibility complex (MHC) or human leukocyte antigen (HLA) or major histocompatibility complex class related protein (MR)1. Accordingly, a heterologous TCR for use in accordance with the invention can be capable of specifically and/or selectively binding, and/or binding with high affinity to AFP, a peptide antigen thereof or peptide antigen comprising the sequence FMNKFIYEI (SEQ ID No: 1), without presentation in complex with a peptide presenting molecule.

Specificity describes the strength of binding between the heterologous TCR and the specific target cancer and/or tumour antigen or peptide antigen thereof and may be described by a dissociation constant, Kd, the ratio between bound and unbound states for the receptor-ligand system. Additionally, the fewer different cancer and/or tumour antigens or peptide antigen thereof the heterologous TCR can bind, the greater its binding specificity. Accordingly, the heterologous TCR may bind, optionally with high affinity, to less than 10, 9, 8, 7, 6, 5, 4, 3, 2 different cancer and/or tumour antigens or peptide antigen thereof.

According to the invention the heterologous TCR may bind or specifically bind, or bind with high affinity, with a dissociation constant of between, 0.01 μM and 100 μM, between 0.01 μM and 50 μM, between 0.01 μM and 20 μM, between 0.05 μM and 10 μMm, between 0.05 μM and 20 μM or of 0.01, 0.02, 0.03, 0.04, 0.05, 0.06, 0.07, 0.08, 0.09, 0.1 μM, 0.15 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1.0 μM, 1.5 μM, 2.0 μM, 2.5 μM, 3.0 μM, 3.5 μM, 4.0 μM, 4.5 μM, 5.0 μM, 5.5 μM, 6.0 μM, 6.5 μM, 7.0 μM, 7.5 μM, 8.0 μM, 8.5 μM, 9.0 μM, 9.5 μM, or 10.0 μM; or between 10 μM and 1000 μM, between 10 μM and 500 μM, between 50 μM and 500 μM or of 10, 20 30, 40, 50 60, 70, 80, 90, 100 μM, 150 μM, 200 μM, 250 μM, 300 μM, 350 μM, 400 μM, 450 μM, or 500 μM; optionally measured with surface plasmon resonance, optionally at 25° C., optionally between a pH of 6.5 and 6.9 or 7.0 and 7.5. The dissociation constant, K_{D} or k_{off}/kₒₙ may be determined by experimentally measuring the dissociation rate constant, k_{off}, and the association rate constant, kₒₙ. A TCR dissociation constant may be measured using a soluble form of the TCR, wherein the TCR comprises a TCR alpha chain variable domain and a TCR beta chain variable domain.

According to the invention the heterologous TCR may bind or specifically bind with a half-life (T½) of between 0.01 and 0.05 sec, of between 0.05 seconds and 0.1 second, of between 0.1 and 0.5 seconds, between 0.5 and 1.0 seconds, between 1 and 1.5 seconds, between 1.5 and 2 seconds, or between 2 and 2.5 seconds.

According to the invention the TCR can have the property of binding the complex of peptide antigen comprising the sequence FMNKFIYEI (SEQ ID No: 1) with HLA-A2 and may have a K_{D} for the complex from about 1 μM to about 21 μM and/or have a binding half-life (T½) for the complex in the range of from about or less than 0.5 seconds to about 2 seconds. It will be appreciated that doubling the affinity of a TCR results in halving the K_{D}. T½ is calculated as In2 divided by the off-rate (k_{off}). So doubling of T½ results in a halving in k_{off}. K_{D} and k_{off} values for TCRs are usually measured for soluble forms of the TCR, i.e. those forms which are truncated to remove cytoplasmic and transmembrane domain residues. In a preferred embodiment these measurements are made using the Surface Plasmon Resonance (BIAcore). K_{D} may be determined by experimentally measuring the dissociation rate constant, k_{off}, and the association rate constant, kₒₙ. The equilibrium constant K_{D} is calculated as k_{off}/kₒₙ.

According to the present invention the TCR binding may be selective for alpha-fetoprotein or peptide antigen thereof in comparison to a closely related cancer and/or tumour antigen or peptide antigen sequence thereof. The closely related cancer and/or tumour antigen or peptide antigen sequence may be of similar or identical length and/or may have a similar number or identical number of amino acid residues. The closely related peptide antigen sequence may share between 50 or 60 or 70 or 80 or 90 to 95 or 98% identity, preferably between 80 to 90% identity and/or may differ by 1, 2, 3 or 4 amino acid residues. The closely related peptide sequence may be derived from the polypeptide sequence of sequence FMNKFIYEI (SEQ ID No: 1). Preferably the TCR binding is at least 10, 20, 30, 40, 50, 60, 70, 80, 90, 100, 250, 500, 750, 1000, 2500, 5000, 7500, or 10000 fold tighter for alpha-fetoprotein or peptide antigen thereof in comparison to the closely related antigen or peptide antigen sequence thereof.

Selective binding denotes that the heterologous TCR binds with greater affinity to one cancer and/or tumour antigen or peptide antigen thereof in comparison to another. Selective binding is denoted by the equilibrium constant for the displacement by one ligand antigen of another ligand antigen in a complex with the heterologous TCR.

According to the invention the modified immunoresponsive cells, for example modified T cells, may be modified to express a heterologous TCR, which binds with increased specificity and/or selectivity and/or affinity to the cancer and/or tumour antigen or peptide antigen thereof, for example to AFP, a peptide antigen thereof or AFP peptide antigen comprising the sequence FMNKFIYEI (SEQ ID No: 1), in comparison to immunoresponsive cells lacking the heterologous TCR or having an alternative heterologous TCR.

The binding affinity may be determined by equilibrium methods (e.g. enzyme-linked immunosorbent assay (ELISA) or radioimmunoassay (RIA)), or kinetics (e.g. BIACORE™ analysis). The TCR binding can also be of high avidity where avidity is the sum total of the strength of binding of two molecules to one another at multiple sites, e.g. taking into account the valency of the interaction. According to the invention the immunoresponsive cells may demonstrate improved affinity and/or avidity to a cancer and/or tumour antigen or peptide antigen thereof, or a cancer and/or tumour antigen or peptide antigen thereof presented by tumour of cancer cell and/or tissue and recognised by the heterologous TCR in comparison to immunoresponsive cells lacking the heterologous TCR or having an alternative heterologous TCR.

According to the invention the modified immunoresponsive cells, for example modified T cells, may be modified to express a heterologous TCR, which binds or specifically binds to tumour cells and/or tissue and/or cancer cells and/or tissue of a subject, patient or cancer patient suffering from a disease condition or cancerous condition, cancer and/or tumour. The subject, patient or cancer patient may be subsequently treated with the modified immunoresponsive cells or modified T cells or population thereof according to the invention. Suitable cancer patients for treatment according to the invention with the modified immunoresponsive cells or modified T cells may be identified by a method comprising; obtaining a sample of tumour and/or cancer cells from an individual or subject with tumour and/or cancer and; identifying the cancer cells as binding to the TCR expressed by the modified immunoresponsive cells.

According to the present invention the heterologous TCR may bind and/or bind specifically and/or selectively bind a peptide presenting molecule for example an HLA presenting or displaying a cancer and/or tumour antigen or peptide antigen thereof, i.e. a peptide fragment of a cancer and/or tumour antigen (pHLA), wherein the HLA corresponds to MHC class I (A, B, and C) which all are the HLA Class1 or specific alleles thereof or the HLA corresponds to MHC class II (DP, DM, DO, DQ, and DR) or specific alleles thereof, preferably the HLA is class 1, preferably the allele is HLA-A*02:01, HLA-A*02:02, HLA-A*02:03, HLA-A*02:04, HLA-A*02:05, HLA-A*02:06, HLA-A*02:642 or HLA-A*02:07, preferably HLA-A*02:01 or HLA-A*02:642. Alternatively, the heterologous TCR may bind and/or bind specifically and/or bind selectively a cancer and/or tumour antigen or peptide antigen thereof, for example AFP or peptide antigen thereof, which is not presented or displayed by HLA.

Preferably, the heterologous TCR is not naturally expressed by the immunoresponsive cells (i.e. the TCR is exogenous or heterologous). A heterologous TCR may include αβTCR heterodimers. A heterologous TCR may be a recombinant or synthetic or artificial TCR i.e. a TCR that does not exist in nature. For example, a heterologous TCR may be engineered to increase its affinity or avidity for a specific cancer and/or tumour antigen or peptide antigen thereof (i.e. an affinity enhanced TCR or specific peptide enhanced affinity receptor (SPEAR) TCR). The affinity enhanced TCR or (SPEAR) TCR may comprise one or more mutations relative to a naturally occurring TCR, for example, one or more mutations in the hypervariable complementarity determining regions (CDRs) of the variable regions of the TCR α and β chains. These mutations may increase the affinity of the TCR for MHCs that display a peptide fragment of a tumour antigen optionally when expressed by tumour and/or cancer cells. Suitable methods of generating affinity enhanced or matured TCRs include screening libraries of TCR mutants using phage or yeast display and are well known in the art (see for example Robbins et al J Immunol (2008) 180(9):6116; San Miguel et al (2015) Cancer Cell 28 (3) 281-283; Schmitt et al (2013) Blood 122 348-256; Jiang et al (2015) Cancer Discovery 5 901). Preferred affinity enhanced TCRs may bind to tumour and/or cancer cells expressing alpha fetoprotein (AFP) or a peptide antigen of AFP or a peptide antigen of AFP comprising FMNKFIYEI (SEQ ID No: 1) or residues 158-166 derived from alpha fetoprotein (AFP) SEQ ID NO: 51.

### Parental TCR

According to the invention the heterologous TCR may be an AFP TCR which may comprise the α chain reference amino acid sequence of SEQ ID NO: 2 or a variant thereof and/or the β chain reference amino acid sequence of SEQ NO: 3 or a variant thereof. A variant may have an amino acid sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference amino acid sequence. The TCR may be encoded by the α chain reference nucleotide sequence of SEQ ID NO: 21 or a variant thereof and the β chain reference nucleotide sequence of SEQ NO: 22 or a variant thereof. A variant may have a nucleotide sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference nucleotide sequence.

According to the invention the heterologous TCR can comprise a TCR alpha chain variable domain and a TCR beta chain variable domain, wherein:
(i) the alpha chain variable domain comprises CDRs having the sequences
DRGSQS (αCDR1), SEQ ID NO:29 or amino acids 27-32 of SEQ ID NO:2, or sequence having at least 50% sequence identity thereto,
IYSNGD (αCDR2), SEQ ID NO:30 or amino acids 50-55 of SEQ ID NO:2, or sequence having at least 50% sequence identity thereto, and
AVNSDSGYALNF (αCDR3), SEQ ID NO:31 or amino acids 90-101 of SEQ ID NO:2, or sequence having at least 50% sequence identity thereto, and
(ii) the beta chain variable domain comprises CDRs having the sequences
SGDLS (βCDR1), SEQ ID NO:32 or amino acids 27-31 of SEQ ID NO:3, or sequence having at least 50% sequence identity thereto,
YYNGEE (βCDR2), SEQ ID NO:33 or amino acids 49-54 of SEQ ID NO:3, or sequence having at least 50% sequence identity thereto, and
ASSLGGESEQY (βCDR3), SEQ ID NO:34 or amino acids 92-102 of SEQ ID NO:3; or sequences having at least 50% sequence identity thereto; optionally the sequence identity may be any of least 50%, at least 55%, at least 60%, at least 65%, at least 70%, at least 75%, at least 80%, at least 85%, at least 90%, at least 95%, at least 98% or at least 99% sequence identity, optionally 100% sequence identity.

According to the invention the heterologous TCR can comprise a TCR alpha chain variable domain and a TCR beta chain variable domain, wherein:
(i) the alpha chain variable domain comprises CDRs having the sequences
DRGSQS (αCDR1), SEQ ID NO:29 or amino acids 27-32 of SEQ ID NO:2,
IYSNGD (αCDR2), SEQ ID NO:30 or amino acids 50-55 of SEQ ID NO:2, and
AVNSDSGYALNF (αCDR3), SEQ ID NO:31 or amino acids 90-101 of SEQ ID NO:2, and
(ii) the beta chain variable domain comprises CDRs having the sequences
SGDLS (βCDR1), SEQ ID NO:32 or amino acids 27-31 of SEQ ID NO:3,
YYNGEE (βCDR2), SEQ ID NO:33 or amino acids 49-54 of SEQ ID NO:3, and
ASSLGGESEQY (βCDR3), SEQ ID NO:34 or amino acids 92-102 of SEQ ID NO:3.

Accordingly, the heterologous TCR may comprise a TCR in which the alpha chain variable domain comprises an amino acid sequence that has at least 70%, 75%, 80%, 85%, 90%, 95%, 96%, 97%, 98% or 99% identity to the sequence of amino acid residues 1-112 of SEQ ID NO:2, and/or the beta chain variable domain comprising an amino acid sequence that has at least 70%, 75%, 80%, 85%, 90%, 95%, 96%, 97%, 98% or 99% identity to the sequence of amino acid residues 1-112 of SEQ ID NO:3. According to the invention the heterologous TCR may comprise an alpha and/or beta chain optionally wherein the alpha chain variable domain comprises an amino acid sequence that has at least 80 or 90% identity to the sequence of amino acid residues 1-112 of SEQ ID No: 2, and/or the beta chain variable domain comprises an amino acid sequence that has at least 80 or 90% identity to the sequence of amino acid residues 1-112 of SEQ ID No: 3. Accordingly, the alpha chain variable domain may have at least 91%, 92%, 93%, 94%, 95%, 96%, 97%, 98%, 99%, or 100% sequence identity to amino acid residues 1 to 112 of SEQ ID No: 2 and/or the beta chain variable domain may have at least 91%, 92%, 93%, 94%, 95%, 96%, 97%, 98%, 99%, or 100% sequence identity to amino acid residues 1 to 112 of SEQ ID No: 3.

According to the invention the heterologous TCR may comprise a TCR in which the alpha chain variable domain comprises amino acid residues 1-112 of SEQ ID No: 2, and the beta chain variable domain comprises amino acid residues 1-112 of SEQ ID No: 3.

According to the invention the heterologous TCR may comprise a TCR comprising an alpha chain TRAC constant domain sequence and/or a beta chain TRBC1 or TRBC2 constant domain sequence. SEQ ID Nos: 2 and 3 are, respectively, the alpha and beta chain extracellular sequences of what is referred to herein as the “parental” AFP TCR. The parental AFP TCR has the following alpha and beta chain usage: Alpha chain: TRAV12-2*02/TRAJ41 *01/TRAC (the extracellular sequence of the parental AFP TCR alpha chain is given in FIG. 1 SEQ ID No: 2. The CDRs are defined by amino acids 27-32 (CDR1), 50-55 (CDR2) and 90-101 (CDR3) of SEQ ID NO: 2; Beta chain: TRBV9*01/TRBD2/TRBJ2-7*01/TRBC2 (the extracellular sequence of the parental AFP TCR alpha chain is given in FIG. 2, (SEQ ID No: 3). The CDRs are defined by amino acids 27-31 (CDR1), 49-54 (CDR2) and 92-102 (CDR3) of SEQ ID NO: 3. The terms ‘*01’ and ‘*02’ indicate there is more than one allelic variant for this sequence, as designated by IMGT nomenclature, and that it is the *01/*02 variant which is present in the TCR clone referred to above. The absence of a “*” qualifier means that only one allele is known for the relevant sequence.

The term “parental TCR”, is used herein to refer to a TCR comprising the AFP TCR α chain and AFP TCR β chain of amino acids 1-112 of SEQ ID NOs: 2 and 3 respectively. It is desirable to provide TCRs that are mutated or modified relative to the parental TCR that have an equal, equivalent or higher affinity and/or an equal, equivalent or slower off-rate for the peptide-HLA complex than the parental TCR. According to the invention the heterologous TCR may have more than one mutation present in the alpha chain variable domain and/or the beta chain variable domain relative to the parental TCR and may be denoted, “engineered TCR” or “mutant TCR”. These mutation(s) may improve the binding affinity and/or specificity and/or selectivity and/or avidity for AFP or peptide antigen thereof. In certain embodiments, there are 1, 2, 3, 4, 5, 6, 7 or 8 mutations in alpha chain variable domain, for example 4 or 8 mutations, and/or 1, 2, 3, 4 or 5 mutations in the beta chain variable domain, for example 5 mutations. In some embodiments, the α chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues amino acids 1-112 of of SEQ ID NO: 2. In some embodiments, the β chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues of amino acids 1-112 of SEQ ID NO: 3.

According to the invention the TCR may comprise a TCR in which, the alpha chain variable domain comprises the amino acid sequence of amino acid residues 1-112 of SEQ ID NO:2, or an amino acid sequence in which amino acid residues 1-26, 33-49, 56-89 and 102-112 thereof have at least 70%, 75%, 80%, 85%, 90%, 95%, 96%, 97%, 98% or 99% identity to the sequence of amino acid residues 1-26, 33-49, 56-89 and 102-112 respectively of SEQ ID NO:2 and/or in which amino acid residues 27-32, 50-55, 90-101, CDR 1, CDR 2, CDR 3 respectively, have at least 70%, 75%, 80%, 85%, 90%, 95%, 96%, 97%, 98% or 99% identity to the sequence of amino acid residues 27-32, 50-55, 90-101, CDR 1, CDR 2, CDR 3, respectively of SEQ ID NO:2.

According to the invention, the TCR may comprise a TCR in which, in the alpha chain variable domain, the sequence of:
(i) amino acid residues 1-26 thereof may have (a) at least 70%, 75%, 80%, 85%, 90% or 95% identity to the sequence of amino acid residues 1-26 of SEQ ID NO:2 or (b) may have one, two or three amino acid residues inserted or deleted relative to residues 1-26 of SEQ ID NO:2,
(ii) amino acid residues 27-32 is DRGSQS αCDR 1, SEQ ID NO:29 or amino acids 27-32 of SEQ ID NO:2,
(iii) amino acid residues 33-49 thereof may have (a) at least 70%, 75%, 80%, 85%, 90% or 95% identity to the sequence of amino acid residues 33-49 of SEQ ID NO: 2 or (b) may have one, two or three amino acid residues inserted or deleted relative to the sequence of amino acid residues 33-49 of SEQ ID NO: 2,
(iv) amino acid residues 50-55 may be IYSNGD αCDR 2, SEQ ID NO:30 or amino acids 50-55 of SEQ ID NO:2,
(v) amino acid residues 56-89 thereof may have at least 70%, 75%, 80%, 85%, 90% or 95% identity to the sequence of amino acid residues 56-89 of SEQ ID NO:2 or may have one, two or three insertions, deletions or substitutions relative to the sequence of amino acid residues 56-89 of SEQ ID NO:2,
(vi) amino acids 90-101 may be AVNSDSGYALNF αCDR 3, SEQ ID NO:31 or amino acids 90-101 of SEQ ID NO:2,
(vii) amino acid residues 102-112 thereof may have at least 70%, 75%, 80%, 85%, 90% or 95% identity to the sequence of amino acid residues 102-112 of SEQ ID NO: 2 or may have one, two or three insertions, deletions or substitutions relative to the sequence of amino acid residues 102-112 of SEQ ID NO: 2.

According to the invention, the TCR may comprise a TCR in which, in the beta chain variable domain comprises the amino acid sequence of amino acid residues 1-112 of SEQ ID NO:3, or an amino acid sequence in which amino acid residues 1-26, 32-48, 55-91, 103-112 thereof have at least 70%, 75%, 80%, 85%, 90% or 95% identity to the sequence of amino acid residues 1-26, 32-48, 55-91, 103-112 respectively of SEQ ID NO:3 and in which amino acid residues 27-31, 49-54 and 92-102 have at least 70%, 75%, 80%, 85%, 90% or 95% identity to the sequence of amino acid residues 27-31, 49-54 and 92-102, βCDR 1, βCDR 2, βCDR 3, respectively of SEQ ID NO:3.

According to the invention, the TCR may comprise a TCR in which, in the beta chain variable domain, the sequence of:
(i) amino acid residues 1-26 thereof may have (a) at least 70%, 75%, 80%, 85%, 90% or 95% identity to the sequence of amino acid residues 1-26 of SEQ ID NO:3 or (b) may have one, two or three amino acid residues inserted or deleted relative to residues 1-26 of SEQ ID NO:3,
(ii) amino acid residues 27-31 is SGDLS, βCDR 1, SEQ ID NO:32 or amino acids 27-31 of SEQ ID NO:3,
(iii) amino acid residues 32-48 thereof may have (a) at least 70%, 75%, 80%, 85%, 90% or 95% identity to the sequence of amino acid residues 32-48 of SEQ ID NO:3 or (b) may have one, two or three amino acid residues inserted or deleted relative to the sequence of amino acid residues 32-48 of SEQ ID NO:3,
(iv) amino acid residues 49-54 may be YYNGEE, βCDR 2, SEQ ID NO:33 or amino acids 49-54 of SEQ ID NO:3,
(v) amino acid residues 55-91 thereof may have at least 70%, 75%, 80%, 85%, 90% or 95% identity to the sequence of amino acid residues 55-91 of SEQ ID NO:3 or may have one, two or three insertions, deletions or substitutions relative to the sequence of amino acid residues 55-91 of SEQ ID NO:3
(vi) amino acids 92-102 may be ASSLGGESEQY, βCDR 3, SEQ ID NO:34 or amino acids 92-102 of SEQ ID NO:3,
(vii) amino acid residues 103-112 thereof may have at least 70%, 75%, 80%, 85%, 90% or 95% identity to the sequence of amino acid residues 103-112 of SEQ ID NO:3 or may have one, two or three insertions, deletions or substitutions relative to the sequence of amino acid residues 103-112 of SEQ ID NO:3.

According to the invention the heterologous TCR may comprise a TCR in which the alpha chain comprises amino acid residues of SEQ ID No: 49, and the beta chain variable domain comprises amino acid residues of SEQ ID No: 3 or SEQ ID NO:50.

### Mutant TCRs

Embodiments of the invention include TCRs which are mutated relative to the parental AFP TCR.

According to the invention the heterologous TCR can comprise an
a. αCDR1 having the sequence DRGSQA, SEQ ID NO:35;
b. αCDR2 having the sequence AVNSDSSYALNF, SEQ ID NO:36;
c. αCDR2 having the sequence AVNSDSGVALNF, SEQ ID NO:37;
d. αCDR1 having the sequence DRGSQA, SEQ ID NO:35 and αCDR2 having the sequence AVNSDSGVALNF, SEQ ID NO:37;
e. αCDR2 having the sequence AVNSQSGYALNF, SEQ ID NO: 38;
f. αCDR2 having the sequence AVNSQSGYSLNF, SEQ ID NO: 39;
g. αCDR2 having the sequence AVNSQSSYALNF, SEQ ID NO: 43;
h. αCDR1 having the sequence DRGSQA, SEQ ID NO:35 and αCDR2 having the sequence AVNSQSGYALNF, SEQ ID NO: 38;
i. αCDR2 having the sequence AVNSQSGVALNF, SEQ ID NO: 39;
j. αCDR2 having the sequence AVNSQNGYALNF, SEQ ID NO: 40;
k. αCDR1 having the sequence DRGSFS, SEQ ID NO: 41;
l. αCDR1 having the sequence DRGSYS, SEQ ID NO: 42;
m. αCDR1 having the sequence DRGSYS, SEQ ID NO: 42 and αCDR2 having the sequence AVNSDSSYALNF SEQ ID NO: 36;
n. αCDR1 having the sequence DRGSYS, SEQ ID NO: 42 and αCDR2 having the sequence AVNSDSSYALNF SEQ ID NO: 36, or
o. αCDR1 having the sequence DRGSYS, SEQ ID NO: 42 and αCDR2 having the sequence AVNSQSGYALNF, SEQ ID NO: 38.

According to the invention the heterologous TCR, or mutated TCR, can comprise an alpha chain variable domain that includes a mutation in one or more of the amino acids corresponding to: 31Q, 32S, 94D, 95S, 96G, 97Y, and 98A, with reference to the numbering shown in SEQ ID No: 2. For example, the alpha chain variable domain may have one or more of the following mutations: Q31F/Y, S32A, D94Q, S95N, G96S, Y97V, A98S, according to the numbering shown in FIG. 1, SEQ ID No: 2.

Accordingly the alpha chain variable domain may comprise an amino acid sequence having at least 90%, 91%, 92%, 93%, 94%, 95%, 96%, 97%, 98%, 99%, or 100% identity to residues 1-112 of any one of SEQ ID No: 6, SEQ ID No: 7, SEQ ID No: 8, SEQ ID No: 9, SEQ ID No: 10, SEQ ID No: 11, SEQ ID No: 12, SEQ ID No: 13, SEQ ID No: 14, SEQ ID No: 15, SEQ ID No: 16, SEQ ID No: 17, SEQ ID No: 18, SEQ ID No: 19 and SEQ ID No: 20, optionally wherein the amino acid sequence also has at least 90% identity to residues 1-112 of SEQ ID No: 2. The amino acids of SEQ ID NO: 6-20 underlined in FIG. 5 may be invariant.

Accordingly, the heterologous TCR may comprise an alpha chain variable domain comprising Q1 to H112 of SEQ ID No: 11, SEQ ID No: 12 or SEQ ID No: 13, and/or a beta chain variable domain comprising D1 to T112 of SEQ ID NO: 3.

According to the invention the heterologous TCR can comprise a TCR alpha chain variable domain and a TCR beta chain variable domain, wherein:
(i) the alpha chain variable domain comprises CDRs having the sequences
DRGSQA (αCDR1), SEQ ID NO:35 or amino acids 27-32 of SEQ ID NO:49,
IYSNGD (αCDR2), SEQ ID NO:30 or amino acids 50-55 of SEQ ID NO:2, and
AVNSQSGYALNF (αCDR3), SEQ ID NO:38 or amino acids 90-101 of SEQ ID NO:49, and
(ii) the beta chain variable domain comprises CDRs having the sequences
SGDLS (βCDR1), SEQ ID NO:32 or amino acids 27-31 of SEQ ID NO:3,
YYNGEE (βCDR2), SEQ ID NO:33 or amino acids 49-54 of SEQ ID NO:3, and
ASSLGGESEQY (βCDR3), SEQ ID NO:34 or amino acids 92-102 of SEQ ID NO:3.

According to the invention the heterologous TCR may comprise a TCR in which the alpha chain variable domain comprises amino acid residues 1-112 of SEQ ID No: 49, and the beta chain variable domain comprises amino acid residues 1-112 of SEQ ID No: 3 or SEQ ID NO:50.

According to the invention the heterologous TCR may comprise a TCR in which the alpha chain comprises amino acid residues of SEQ ID No: 49, and the beta chain variable domain comprises amino acid residues 1-112 of SEQ ID No: 3 or SEQ ID NO:50.

### Soluble TCRS

For the purpose of providing a reference TCR against which the binding profile of mutated TCRs of the invention may be compared, it is convenient to use the soluble TCR having the extracellular sequence of the AFP TCR alpha chain given in FIG. 3 (SEQ ID No: 4) and the extracellular sequence of the AFP TCR beta chain given in FIG. 4 (SEQ ID No: 5). That TCR is referred to herein as the “the reference TCR” or “the reference AFP TCR”. Note that SEQ ID No: 4 is identical to the parental alpha chain extracellular sequence SEQ ID No: 2 except that C159 has been substituted for T159 (i.e. T48 of TRAC). Likewise, SEQ ID No: 5 is identical to the parental beta chain extracellular sequence SEQ ID No: 3 except that C169 has been substituted for S169 (i.e. S57 of TRBC2), A187 has been substituted for C187 and D201 has been substituted for N201. These cysteine substitutions relative to the parental AFP alpha and beta chain extracellular sequences enable the formation of an interchain disulfide bond which stabilises the refolded soluble TCR, ie the TCR formed by refolding extracellular alpha and beta chains. Use of the stable disulfide linked soluble TCR as the reference TCR enables more convenient assessment of binding affinity and binding half-life.

Hence, according to the invention the heterologous TCR, can comprise an alpha and/or beta chain constant domain sequence(s) which are modified by truncation or substitution to delete the native disulfide bond between Cys4 of exon 2 of TRAC and Cys2 of exon 2 of TRBC1 or TRBC2; or wherein the alpha and/or beta chain constant domain sequence(s) are modified by substitution of cysteine residues for Thr 48 of TRAC and Ser 57 of TRBC1 or TRBC2, the cysteines forming a disulfide bond between the alpha and beta constant domains of the TCR.

### Variant Tcrs

Also within the scope of the present invention are phenotypically silent variants of any TCR disclosed herein. As used herein the term “phenotypically silent variants” is understood to refer to those TCRs which have a K_{D} and/or binding half-life for AFP peptide antigen for example the FMNKFIYEI (SEQ ID No: 1) optionally as an HLA-A2 complex within the ranges of K_{D}s and binding half-lives detailed above. For example, as is known to those skilled in the art, it may be possible to produce TCRs that incorporate changes in the constant and/or variable domains thereof compared to those detailed above without altering the affinity for the interaction with AFP peptide antigen for example the FMNKFIYEI (SEQ ID No: 1) optionally as HLA-A2 complex. Such trivial variants are included in the scope of this invention. Those TCRs in which one or more conservative substitutions have been made also form part of this invention.

Amino acid and nucleotide sequence identity is generally defined with reference to the algorithm GAP (GCG Wisconsin Package™, Accelrys, San Diego Calif.). GAP uses the Needleman & Wunsch algorithm (J. Mol. Biol. (48): 444-453 (1970)) to align two complete sequences that maximizes the number of matches and minimizes the number of gaps. Generally, the default parameters are used, with a gap creation penalty=12 and gap extension penalty=4. Use of GAP may be preferred but other algorithms may be used, e.g. BLAST, psiBLAST or TBLASTN (which use the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), generally employing default parameters.

Particular amino acid sequence variants may differ from a reference sequence by insertion, addition, substitution or deletion of 1 amino acid, 2, 3, 4, 5-10, 10-20 or 20-30 amino acids. In some embodiments, a variant sequence may comprise the reference sequence with 1, 2, 3, 4, 5, 6, 7, 8, 9, 10 or more residues inserted, deleted or substituted. For example, up to 15, up to 20, up to 30 or up to 40 residues may be inserted, deleted or substituted.

In some preferred embodiments of the present invention, a variant TCR may differ from a reference TCR sequence by 1, 2, 3, 4, 5, 6, 7, 8, 9, 10 or more conservative substitutions. Conservative substitutions involve the replacement of an amino acid with a different amino acid having similar properties. For example, an aliphatic residue may be replaced by another aliphatic residue, a non-polar residue may be replaced by another non-polar residue, an acidic residue may be replaced by another acidic residue, a basic residue may be replaced by another basic residue, a polar residue may be replaced by another polar residue or an aromatic residue may be replaced by another aromatic residue. Conservative substitutions may, for example, be between amino acids within the following groups:
(i) alanine and glycine;
(ii) glutamic acid, aspartic acid, glutamine, and asparagine
(iii) arginine and lysine;
(iv) asparagine, glutamine, glutamic acid and aspartic acid
(v) isoleucine, leucine and valine;
(vi) phenylalanine, tyrosine and tryptophan
(vii) serine, threonine, and cysteine.
(viii) As will be obvious to those skilled in the art, it may be possible to truncate the sequences provided at the C-terminus and/or N-terminus thereof, by 1, 2, 3, 4, 5 or more residues, without substantially affecting the binding characteristics of the TCR. All such trivial variants are encompassed by the present invention. Native TCRs exist in heterodimeric αβ or γδ forms. However, recombinant TCRs consisting of αα or ββ homodimers have previously been shown to bind to peptide MHC molecules. Therefore, the TCR of the invention may be a heterodimeric αβ TCR or may be an αα or ββ homodimeric TCR.
(ix) For use in adoptive therapy, an ap heterodimeric TCR may, for example, be transfected as full length chains having both cytoplasmic and transmembrane domains. In certain embodiments TCRs of the invention may have an introduced disulfide bond between residues of the respective constant domains, as described, for example, in WO 2006/000830.
(x) TCRs of the invention, particularly alpha-beta heterodimeric TCRs, may comprise an alpha chain TRAC constant domain sequence and/or a beta chain TRBC1 or TRBC2 constant domain sequence. The alpha and beta chain constant domain sequences may be modified by truncation or substitution to delete the native disulfide bond between Cys4 of exon 2 of TRAC and Cys2 of exon 2 of TRBC1 or TRBC2. The alpha and/or beta chain constant domain sequence(s) may also be modified by substitution of cysteine residues for Thr 48 of TRAC and Ser 57 of TRBC1 or TRBC2, the said cysteines forming a disulfide bond between the alpha and beta constant domains of the TCR.
(xi) TCRs of the invention may be in single chain format, for example see WO 2004/033685. Single chain formats include αβ TCR polypeptides of the Vα-L-vβ, vβ-L-Vα, Vα-Cα-L-Vβ, Vα-L-Vβ-Cβ, Vα-Cα-L-Vβ-Cβ types, wherein Vα and Vβ are TCR α and p variable regions respectively, Cα and Cβ are TCR α and β constant regions respectively, and L is a linker sequence. In certain embodiments single chain TCRs of the invention may have an introduced disulfide bond between residues of the respective constant domains, as described in WO 2004/033685.

### Heterologous TCR

According to the present invention the modified immunoresponsive cells can express a heterologous T cell receptor (TCR). Upon binding to the antigen and/or antigenic peptide thereof (such as AFP or peptide antigen thereof), the modified immunoresponsive cells can exhibit T cell effector functions and/or cytolytic effects towards cells bearing the antigen (e.g. AFP) and/or antigenic peptide thereof and/or undergo proliferation and/or cell division. In certain embodiments, the modified immunoresponsive cells comprising the TCR exhibits comparable or better therapeutic potency compared to cells comprising a chimeric antigen receptor (CAR) targeting the same cancer and/or tumour antigen (e.g. AFP) and/or antigenic peptide thereof. Activated modified immunoresponsive cells comprising the TCR can secrete anti-tumour cytokines which can include, but are not limited to, TNFalpha, IFNy and IL2.

According to the invention the modified immunoresponsive cells may comprise a nucleic acid, construct or vector, or heterologous nucleic acid, construct or vector, encoding the heterologous T cell receptor (TCR). Optionally the TCR may be an affinity enhanced TCR, for example a specific peptide enhanced affinity receptor (SPEAR) TCR.

The term “heterologous” or “exogenous” refers to a polypeptide or nucleic acid that is foreign to a particular biological system, such as a cell or host cell, for example immunoresponsive cell, and is not naturally present in that system and which may be introduced to the system by artificial or recombinant means. Accordingly, the expression of a TCR which is heterologous, may thereby alter the immunogenic specificity of the T cells so that they recognise or display improved recognition for one or more tumour or cancer antigens (e.g. AFP) and/or antigenic peptides thereof that are present on the surface of the cancer cells of an individual with cancer. The modification of T cells and their subsequent expansion may be performed in vitro and/or ex vivo.

### The CD8α Co-Receptor

According to the present invention, the population of modified immunoresponsive cells expressing or presenting a heterologous TCR may further express or present a heterologous co-receptor. The heterologous co-receptor may be a CD8 co-receptor. The CD8 co-receptor may comprise a dimer or pair of CD8 chains which comprises a CD8-α and CD8-β chain or a CD8-α and CD8-α chain. Preferably, the CD8 co-receptor is a CD8αα co-receptor comprising a CD8-α and CD8-α chain. A CD8a co-receptor may comprise the amino acid sequence of at least 80% identity to SEQ ID NO: 47 or a variant thereof, or 100% identity SEQ ID NO: 47 or a variant thereof. The CD8a co-receptor may be a homodimer.

The CD8 co-receptor binds to class 1 MHCs and potentiates TCR signalling. According to the invention the CD8 co-receptor may comprise the reference amino acid sequence of SEQ ID NO: 47 or amino acids 22-235 of SEQ ID NO: 47 or may be a variant thereof. A variant may have an amino acid sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference amino acid sequence SEQ ID NO: 47 or amino acids 22-235 of SEQ ID NO: 47. The CD8 co-receptor may be encoded by the reference nucleotide sequence of SEQ ID NO: 48 or may be a variant thereof. A variant may have a nucleotide sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference nucleotide sequence SEQ ID NO: 48. Optionally the CD8 co-receptor may comprise CDRs having the sequence;
(i) VLLSNPTSG, CDR1, SEQ ID NO: 44, or amino acids 45-53 of SEQ ID NO: 47,
(ii) YLSQNKPK, CDR2, SEQ ID NO: 45 or amino acids 72-79 of SEQ ID NO: 47,
(iii) LSNSIM, CDR3, SEQ ID NO: 46 or amino acids 80-117 of SEQ ID NO: 47,

or sequences having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity thereto.

According to the invention the heterologous CD8 co-receptor may comprise a CD8 co-receptor in which, in the Ig like V-type domain comprises CDRs having the sequence;
(i) VLLSNPTSG, CDR1, SEQ ID NO: 44, or amino acids 45-53 of SEQ ID NO: 47,
(ii) YLSQNKPK, CDR2, SEQ ID NO: 45 or amino acids 72-79 of SEQ ID NO: 47,
(iii) LSNSIM, CDR3, SEQ ID NO: 46 or amino acids 80-117 of SEQ ID NO: 47,
   or sequences having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity thereto.

According to the invention the heterologous CD8 co-receptor may comprise a CD8 co-receptor which comprises or in which, in the Ig like V-type domain comprises, residues 22-135 of the amino acid sequence of SEQ ID No:47, or an amino acid sequence in which amino acid residues 22-44, 54-71, 80-117, 124-135 thereof have at least 70%, 75%, 80%, 85%, 90% or 95% identity to the sequence of amino acid residues 22-44, 54-71, 80-117, 124-135, respectively of SEQ ID No:47 and in which amino acid residues 45-53, 72-79 and 118-123 have at least 70%, 75%, 80%, 85%, 90% or 95% identity to the sequence of amino acid residues 45-53, 72-79 and 118-123 respectively of SEQ ID No:47.

According to the invention the CD8 co-receptor may comprise a CD8 co-receptor in which, or in which in the Ig like V-type domain, the sequence of:
(i) amino acid residues 22-44 thereof may have (a) at least 70%, 75%, 80%, 85%, 90% or 95% identity to the sequence of amino acid residues 22-44 of SEQ ID NO:47 or (b) may have one, two or three amino acid residues inserted or deleted relative to residues 22-44 of SEQ ID NO:47,
(ii) amino acid residues 45-53 is VLLSNPTSG, SEQ ID NO:44, CDR1, or amino acids 45-53 of SEQ ID NO:47,
(iii) amino acid residues 54-71 thereof may have (a) at least 70%, 75%, 80%, 85%, 90% or 95% identity to the sequence of amino acid residues 54-71 of SEQ ID NO:47 or (b) may have one, two or three amino acid residues inserted or deleted relative to the sequence of amino acid residues 54-71 of SEQ ID NO:47,
(iv) amino acid residues 72-79 may be YLSQNKPK, CDR2, SEQ ID NO:45 or amino acids 72-79 of SEQ ID NO:47,
(v) amino acid residues 80-117 thereof may have at least 70%, 75%, 80%, 85%, 90% or 95% identity to the sequence of amino acid residues 80-117 of SEQ ID NO:47 or may have one, two or three insertions, deletions or substitutions relative to the sequence of amino acid residues 80-117 of SEQ ID NO:47;
(vi) amino acids 118-123 may be LSNSIM, CDR3, SEQ ID NO:46 or amino acids 80-117 of SEQ ID NO:47,
(vii) amino acid residues 124-135 thereof may have at least 70%, 75%, 80%, 85%, 90% or 95% identity to the sequence of amino acid residues 124-135 of SEQ ID NO:47 or may have one, two or three insertions, deletions or substitutions relative to the sequence of amino acid residues 124-135 of SEQ ID NO:47.

The modified immunoresponsive cells that express heterologous CD8 co-receptor may demonstrate improved affinity and/or avidity and/or improved T-cell activation, as determinable by the assays disclosed herein, towards or on stimulation by antigenic peptide, tumour or cancer antigen optionally when presented on HLA relative to modified immunoresponsive cells that do not express heterologous CD8 co-receptor. The heterologous CD8 of modified immunoresponsive cells may interact or bind specifically to an MHC, the MHC may be class I or class II, preferably class I major histocompatibility complex (MHC), HLA-I molecule or with the MHC class I HLA-A/B2M dimer, preferably the CD8-α interacts with the α₃ portion of the Class I MHC (between residues 223 and 229), preferably via the IgV-like domain of CD8. Accordingly the heterologous CD8 improves TCR binding of the immunoresponsive cells to the HLA and/or antigenic peptide bound or presented by HLA pMHCl or pHLA, optionally on the surface of antigen presenting cell, dendritic cell and/or tumour or cancer cell, tumour or cancer tissue compared to immunoresponsive cells lacking the heterologous CD8. Accordingly the heterologous CD8 can improve or increase the off-rate (k_{off}) of the cell (TCR)/peptide-major histocompatibility complex class I (pMHCI) interaction of the immunoresponsive cells, and hence its half-life, optionally on the surface of antigen presenting cell, dendritic cell and/or tumour or cancer cell, or tumour or cancer tissue compared to the cells lacking the heterologous CD8, and thereby may also provide improved ligation affinity and/or avidity. The heterologous CD8 can improve organizing the TCR on the immunoresponsive cell surface to enable cooperativity in pHLA binding and may provide improved therapeutic avidity. Accordingly, the heterologous CD8 co-receptor modified immunoresponsive cells may bind or interact with LCK (lymphocyte-specific protein tyrosine kinase) in a zinc-dependent manner leading to activation of transcription factors like NFAT, NF-κB, and AP-1.

According to the invention the modified immunoresponsive cells may have an improved or increased expression of CD40L, cytokine production, cytotoxic activity, induction of dendritic cell maturation or induction of dendritic cell cytokine production, optionally in response to cancer and/or tumour antigen or peptide antigen thereof optionally as presented by tumour of cancer cell or tissue, in comparison to immunoresponsive cells lacking the heterologous CD8 co-receptor.

### Co-Stimulatory Ligand

According to the present invention, the modified immunoresponsive cells, may further comprise an exogenous or a recombinant (e.g., the cell is transduced with) at least one co-stimulatory ligand, optionally one, two, three or four. The modified immunoresponsive cells, may co-express the heterologous TCR and the at least one exogenous co-stimulatory ligand. The interaction between the heterologous TCR and at the least one exogenous co-stimulatory ligand may provide a non-antigen-specific signal and activation of the cell. Co-stimulatory ligands include, but are not limited to, members of the tumour necrosis factor (TNF) superfamily, and immunoglobulin (Ig) superfamily ligands. TNF is a cytokine involved in systemic inflammation and stimulates the acute phase reaction. TNF superfamily members include, but are not limited to, nerve growth factor (NGF), CD40L (CD40L)/CD154, CD137L/4-1BBL, TNF-alpha, CD134L/OX40L/CD252, CD27L/CD70, Fas ligand (FasL), CD30L/CD153, tumour necrosis factor beta (TNFP)/lymphotoxin-alpha (LTa), lymphotoxin-beta (TTb), CD257/B cell-activating factor (BAFF)/Blys/THANK/Tall-I, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28. In certain embodiments, the at least one co-stimulatory ligand is selected from the group consisting of 4-1 BBL, CD275, CD80, CD86, CD70, OX40L, CD48, TNFRSF14, and combinations thereof. The at least one exogenous or recombinant co-stimulatory ligand can be 4-1 BBL or CD80, preferably, the at least one exogenous or recombinant co-stimulatory ligand is 4-1 BBL. The modified immunoresponsive cells may comprise two exogenous recombinant co-stimulatory ligands, preferably the two exogenous or recombinant co-stimulatory ligands are 4-1 BBL and CD80.

The modified immunoresponsive cells may comprise an exogenous or a recombinant (e.g., the cell is transduced with) at least one construct which overcomes the immunosuppressive tumour microenvironment. Such constructs can be, but are not limited to, cyclic AMP phosphodiesterases and dominant-negative transforming growth factor beta (TGFbeta) receptor II. The modified immunoresponsive cell, modified T cell or a population of modified immunoresponsive cells for example T cells may be engineered to release cytokines which have a positive effect on the cytolytic activity of said cells. Such cytokines include, but are not limited to interleukin-7, interleukin-15 and interleukin-21.

### Immunoresponsive Cells

According to the present invention the modified immunoresponsive cells can be cells of the lymphoid lineage, comprising B, T or natural killer (NK) cells. The modified immunoresponsive cells may be cells of the lymphoid lineage including T cells, Natural Killer T (NKT) cells, and precursors thereof including embryonic stem cells, and pluripotent stem cells (e.g, those from which lymphoid cells may be differentiated). T cells can be lymphocytes that mature in the thymus and are chiefly responsible for cell-mediated immunity and also involved in the adaptive immune system. According to the present invention the T cells can include, but are not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), Natural killer T cells, Mucosal associated invariant T cells, and gamma-delta T cells. Cytotoxic T cells (CTL or killer T cells) are a subset of T-lymphocytes capable of inducing the death of infected somatic or tumour cells. A subject's own T cells may be genetically modified to target specific antigens through the introduction of a heterologous TCR. Preferably, the modified immunoresponsive cell is a T cell optionally a CD4⁺T cell or a CD8⁺T cell. Accordingly the modified immunoresponsive cells may be T-cells, optionally CD4+ T cells or CD8+ T cells, or the modified immunoresponsive cells may be a population of modified T-cells, optionally CD4+ T cells; or CD8+ T cells, or a mixed population of CD4+ T cells and CD8+ T cells.

### Therapy

### AFP Expression in Serum

The present invention and the methods, treatment and uses of the present invention provides a reduction in serum AFP expression or concentration compared to the pre-treatment serum AFP expression or concentration or in comparison to without treatment or in comparison to treatment comprising a standard of care.

Changes in serum AFP levels from Baseline (pre-treatment) are correlated with response to treatment and correspond to tumour AFP expression from tissue biopsies and indicates treatment efficacy and success of cancer and/or tumour treatment.

Accordingly the present invention and the methods, treatment and uses of the present invention provides a reduction in serum AFP expression or concentration in comparison to placebo treatment or in comparison to without treatment or compared to pre-treatment, or in comparison to treatment comprising a standard of care, optionally wherein the standard of care treatment is selected from any one of Sorafenib, a PD1 or PD-L1 antagonist or inhibitor, Regorafenib, Cabozantinib, Sunitinib, Brivanib, Everolimus, Tivantinib, Linifanib.

### Serum Cytokine and Soluble Factor Analysis and T-Cell Infiltration of Tumour

The present invention and the methods, treatment and uses of the present invention provides an increase in serum cytokine and/or interferon level or concentration compared to the pre-treatment serum cytokine and/or interferon level or concentration or in comparison to without treatment or treatment comprising a standard of care as hereinabove described.

Accordingly the invention provides an improved or enhanced cancer and/or tumour immunogenicity, for example as measured by the ability to provoke an immune response in response to tumour or tumour antigen, for example enhanced by at least 10%, alternatively 20%, 30%, 40%, 50%, 60%, 70%, 80%, 90%, 100%, 120%, 150%, 200% or more relative to such levels before the treatment or intervention or compared to placebo, or relative to without treatment or relative to treatment comprising a standard of care, for example as judged by increased secretion of cytokines and/or interferon, increased T-cell proliferation, increased antigen responsiveness, target cell killing, T-cell activation, CD28 signalling, T-cell infiltration of tumour, ability to recognise and bind to dendritic cell presented antigen.

The efficacy of immunotherapy of cancer is conditioned by the infiltration of tumours by activated tumour-specific T-cells. The activity of these T-cells will in turn be affected by the presence in the tumour of an immunosuppressive environment (e.g. regulatory T-cells).

Therefore, the direct evaluation of the “immune landscape” inside the tumour is of great value for monitoring efficacy of the T-cell immunotherapy and may be quantitated by tumour biopsies to evaluate the immune status of the tumour before and after T-cell infusion. Accordingly the invention provides an improved T-cell infiltration of tumour and/or reduction in T-cell repressive factors as determined for example by a reduction in level of T-regs, Myeloid derived suppressor cells (MDSCs), PD-L1 protein expression, serum cytokine levels selected from CCL3, IL8, IL1β, CXCL10, or sIL2Rα or levels of inhibitory receptors, selected from PD-1, CTLA-4, TIM-3, LAG-3, BTLA or TIGIT compared to pre-treatment (e.g. prior to treatment or before treatment according to the invention) or without treatment or in comparison to treatment comprising a standard of care. Alternatively as determined from the increase in level of interferon-γ, interleukin-6, interleukin-10, cytokine production, such as IL-2, TNF-α, IFN-γ and granzyme B or innate immune cells such as NK cells, adaptive immune cells (CD4⁺ and CD8⁺) or improved proliferation in T-cells for example as judged by Ki67 expression level, compared to pre-treatment or without treatment or in comparison to treatment comprising a standard of care as described herein.

According to the foregoing, the standard of care treatment may be selected from any one of Sorafenib, a PD1 or PD-L1 antagonist or inhibitor, Regorafenib, Cabozantinib, Sunitinib, Brivanib, Everolimus, Tivantinib, Linifanib.

### Tumour Size and Tumour Burden

The present invention and the methods, treatment and uses of the present invention provides an improved or enhanced level or response of reducing tumour growth or tumour growth rate or maintaining tumour size after cessation of treatment or of tumour number or tumour burden, in comparison to prior to treatment or without treatment or treatment comprising a standard of care, for example, as determined by the measurement of tumour size or tumour number, preferably improved or enhanced by at least 10%, alternatively 20%, 30%, 40%, 50%, 60%, 70%, 80%, 90%, 100%, 120%, 150%, 200% or more relative to prior to treatment or without treatment or treatment comprising a standard of care. Preferably an improved or enhanced level or response may be a sustained improved or enhanced level or response and/or may have a duration at least the same as the treatment duration, at least 1.5, 2.0, 2.5, or 3.0 or more times the length of the treatment duration. Such improved or enhanced level or response may be judged from RECIST 1.1 measurements [E. A. Eisenhauer., et. al., EUROPEAN JOURNAL OF CANCER 45 (2009) 228-247] or by tumour biopsy or liquid biopsy (plasma from peripheral blood) to determine-free DNA (cfDNA) or exosomes (source of stable mRNA). Exosomes (produced by all cells, including tumor cells and immune cells) and cfDNA (produced by dying tumor cells) may be used to monitor both the tumour burden and the immune response. The analysis of exosomes and cfDNA may allow: (a) estimation and genetic profiling of the global tumour burden (including expression of AFP mRNA and mutational profiling) from exosomes and cfDNA, (b) Systemic assessment of the immune response (gene expression by cytotoxic and regulatory immune cells) from exosomes.

According to the foregoing, the standard of care treatment may be selected from any one of Sorafenib, a PD1 or PD-L1 antagonist or inhibitor, Regorafenib, Cabozantinib, Sunitinib, Brivanib, Everolimus, Tivantinib, Linifanib.

### 7.5.5. AFP TCR+ Cell Persistence

The present invention and the methods, treatment and uses of the present invention provides improved therapeutic effect and improved treatment, prevention or delaying in the progression of cancer and/or tumour in a subject, in comparison to prior to treatment or without treatment or treatment comprising a standard of care, for example, as determined by the measurement of the persistence of infused engineered and modified immunoresponsive cells expressing or presenting a heterologous T-cell receptor (TCR). Persistence of the infused engineered and modified immunoresponsive cells is correlated with therapeutic effect and is also a long-term safety measure. Cell persistence can be determined by qPCR or flow cytometry (FCM). For example the quantitation of AFP TCR+ cells by PCR of transgene from DNA extracted from frozen PBMC may be used as a measure, likewise the quantitation of AFP TCR expressing cells by FCM from frozen PBMC. T cell phenotype and activity may be determined by a range of assays, for example:
Phenotype analysis for determination of T-cell lineages in cell product and in the blood post infusion.
Quantitation of the senescence and activation status of T-cells from PBMC.
Quantitation of soluble factors reflecting in vivo function of infused T cells, for example AFP TCR+ T-cells.
Ex-vivo activity of transduced cells at different time points to assess potential functionality of those cells.

### T-Cell Function

The present invention and the methods, treatment and uses of the present invention provides an enhancement of T-cell function compared to pre-treatment or in comparison to without treatment or in comparison to treatment comprising a standard of care. Preferably the T-cell function is enhanced by at least 10%, alternatively 20%, 30%, 40%, 50%, 60%, 70%, 80%, 90%, 100%, 120%, 150%, 200% or more, for example as judged by increased secretion of y-interferon from CD8+ T-cells, increased T-cell proliferation, increased internal signalling, increased antigen responsiveness, increased secretion of cytokines and/or interferon, increased target cell killing, increased T-cell activation, increased CD28 signalling, increased T-cell ability to infiltrate tumour, or increased ability to recognise and bind to dendritic cell presented antigen.

According to the present invention and the methods and uses of the present invention, tumour immunity or evasion of immune recognition by the tumour may be attenuated resulting in improved tumour recognition and attack by the immune system and thereby treating tumour immunity for example as measured by tumour binding, tumour shrinkage and tumour clearance. Accordingly, the present invention provides treatment of tumour immunity and/or provides treatment of tumour immunity which is enhanced by at least 10%, alternatively 20%, 30%, 40%, 50%, 60%, 70%, 80%, 90%, 100%, 120%, 150%, 200% or more compared to pre-treatment (e.g. prior to treatment or before treatment according to the invention) or in comparison to without treatment or in comparison to treatment comprising a standard of care as herein described, for example as measured by tumour binding, tumour shrinkage and tumour clearance.

In the context of T-cell activity the term “dysfunction” refers to a state of reduced immune responsiveness to antigenic stimulation and includes T-cell exhaustion and/or anergy whereby the T-cell may recognise and bind antigen but shows reduced effectiveness in progressing immune response or combating tumour growth. Dysfunctional T-cells demonstrate impaired capacity to translate antigen recognition into down-stream T-cell effector functions, such as proliferation, cytokine and interferon production or target cell killing and/or appear refractory or unresponsive to antigen recognition as is characteristic of T-cell dysfunctional disorder. “T-cell dysfunctional disorder” may be associated with or detected as inappropriate increased T-cell signalling through PD-1; T-cells having decreased ability to proliferate and/or produce cytokines and/or cytolytic activity; T-cell anergy; tumour immunity.

“T-cell exhaustion” comprises a state of T cell dysfunction due to sustained TCR signalling as part of the response to cancer and prevents optimal response to tumours. Exhaustion can find effect through either the cell intrinsic negative regulatory (costimulatory) pathways (for example PD-1, PD-1 axis, B7-H3, B7-H4) or through the cell extrinsic negative regulatory pathways (immunoregulatory cytokines). T-cell exhaustion is characterised by poor effector function, sustained expression of inhibitory receptors and an altered activity of transcription distinct from that of functional effector or memory T-cells. T-cell anergy occurs through deficient signalling through the T-cell receptor and a resulting state of unresponsiveness to antigen stimulation often even in the context of costimulation, consequently such T-cells do not undergo clonal expansion and/or acquire effector functions.

### Treatment Administration

According to the invention the modified immunoresponsive cells may be administered continuously or intermittently, optionally as a single dose or as more than one dose.

Accordingly the modified immunoresponsive cells may be administered as a single dose or as more than one dose (multiple doses). The modified immunoresponsive cells may be administered at a dose of between about 500 million to any one of about 1 billion cells, about 2 billion cells, about 3 billion cells, about 4 billion cells, about 5 billion cells, about 6 billion cells, about 7 billion cells, about 8 billion cells, about 9 billion cells, about 10 billion cells, about 11 billion cells, about 12 billion cells, about 13 billion cells, about 14 billion cells, about billion cells, about 16 billion cells, about 17 billion cells, about 18 billion cells, about 19 billion cells, about 20 billion cells, or about 21 billion cells. The modified immunoresponsive cells may be administered at a dose of between about 100 million to about 200 million cells, about 300 million to about 400 million cells, about 500 million to about 600 million cells, about 700 million to about 800 million cells, or about 900 million to about 1 billion cells, optionally about 500 million to about 1 billion cells, about 2 billion to about 5 billion cells or about 6 billion to about 10 billion cells.

According to the invention the modified immunoresponsive cells may be administered, intravenously, intramuscularly, subcutaneously, topically, orally, transdermally, intraperitoneally, intraorbitally, by implantation, by inhalation, intrathecally, intraventricularly, or intranasally or by intravenous infusion. Preferably, the modified immunoresponsive cells may be administered intravenously or by intravenous infusion.

According to the invention modified immunoresponsive cells can be administered as
(a) a single dose in each of one or more dosing cycles,
(b) one or more doses in each of one or more dosing cycles,
(c) a single dose on the first day of each of one or more dosing cycles,
(d) one or more doses in each of one or more dosing cycles comprising a dose on the first day of each of the one or more dosing cycles,
(e) one or more doses in each of one or more dosing cycles, at least one dose being on the first day of each cycle,
(f) a single dose.

According to the invention modified immunoresponsive cells can be administered in a dosing cycle wherein the dosing cycle can be any of 12, 13, 14, 15, 16, 17, 18, 19, 20, 21, 22, 23, 24, 25, 26, 27, 28 weeks or any of 1 month, 2 months, 3 months, 4 months, 5 months, 6 months, 7 months, 8 months, 9 months, 10 months, 11 months, or 12 months. Accordingly the dosing cycle can be any of 10 to 12 weeks, 11 to 13 weeks, 14 to 17 weeks, 14 to 17 weeks, 18 to 21 weeks, 22 to 24 weeks, 24 to 27 weeks, 28 to 30 weeks, 3 months, 4 months, 5 months, 6 months.

According to the invention modified immunoresponsive cells can be administered in a dosing cycle wherein the dosing cycle can be on, or commence on or re-commence on:
(a) disease progression following a previous administration of modified immunoresponsive cells, and/or
(b) 12 weeks or more following the previous administration of modified immunoresponsive cells, and wherein
(c) the tumour and/or cancer expresses AFP and/or
(d) the subject serum AFP is above the normal range.

According to the invention modified immunoresponsive cells can be administered in a dosing cycle wherein the dosing cycle can be on, or commence on or re-commence on:
(a) confirmed response or complete response or partial response following a previous administration of modified immunoresponsive cells, or (b) stable disease for a period of greater than or equal to 2, 3, or 4 months; followed by disease progression following the previous administration of modified immunoresponsive cells, and/or
(c) greater than or equal to 12 weeks following the previous administration of modified immunoresponsive cells, and wherein
(c) the tumour and/or cancer expresses AFP and/or
(d) the subject serum AFP is above the normal range.

The tumour and/or cancer may express AFP at a level greater than or equal to an intensity of 1+ in greater than or equal to 15, 16, 17, 18, 19, 20, 21, 22, 23, 24, 25, 26, 27, 28, 29 or 30%, preferably greater than or equal to 20% of tumour and/or cancer cells as determined by immunohistochemistry. The subject serum AFP above the normal range may be greater than or equal to 50, 60, 70, 80, 90, 100, 150, 200, 250, 300, 350, 400, 450 or 500 ng/mL, preferably greater than or equal to 100 ng/mL.

According to the invention the dose may be a fixed dose or a variable dose. For example where more than one dose is administered, i.e. multiple dose, the dose may be fixed or may be variable, for example where more than one dose is administered the dose may be escalated or increased, for example in each dosing cycle, i.e. may be of increasing level of dose, for example in progression, for example 100 million to 500 million to 1 billion to 5 billion to 10 billion cells.

According to the invention the modified immunoresponsive cells are preferably administered as a single dose of between about 5 billion and about 10 billion cells.

According to the invention the modified immunoresponsive cells can be administered for a specified period, meaning that the modified immunoresponsive cells dosing cycles can administered for a specified period. The specified period may be any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 months, preferably 24 months.

According to the invention the method may comprise the steps wherein
(a) the modified immunoresponsive cells is administered as a single dose,
(b) the status of disease is determined at a period after the modified immunoresponsive cells administration and compared to the status prior to the modified immunoresponsive cells administration, wherein if progressive disease is determined then,
(c) modified immunoresponsive cells are administered as a single dose, optionally wherein the tumour and/or cancer expresses AFP and/or the subject serum AFP is above the normal range as hereinbefore described. Preferably the period is greater than or equal to any one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 weeks, preferably greater than or equal to 12 weeks

According to the invention the method may comprise the steps wherein
(a) the modified immunoresponsive cells is administered as a single dose,
(b) the status of disease is determined at a first and a later second period after the modified immunoresponsive cells administration and compared to the status prior to the modified immunoresponsive cells administration, wherein if stable disease is determined after the first period and progressive disease is determined after the second period then,
(c) modified immunoresponsive cells is administered as a single dose, optionally wherein the tumour and/or cancer expresses AFP and/or the subject serum AFP is above the normal range as hereinbefore described. Preferably the first period is greater than or equal to any one of 1, 2, 3, 4, 5, 6, 7, 8, months, preferably greater than or equal to 4 months.

According to the invention a “complete response” (CR) is determined where all target lesions or tumours have been assessed or measured as having disappeared. “Partial response” (PR) is determined when there is a measurement of an at least a 30% decrease in the sum of the longest diameters (SLD) of target lesions or tumours, for example as referenced to the control or pre-treatment comparator. “Progressive disease” (PD) is determined when there is a measurement of at least a 20% increase in the sum of the longest diameters (SLD) of target lesions or tumours, for example as referenced to the control or pre-treatment comparator, since the treatment started or the presence of one or more new lesions. “Stable disease” (SD) is determined where it is determined that there is neither sufficient reduction or decrease in the sum of the longest diameters (SLD) of target lesions or tumours to qualify for PR, nor sufficient increase to qualify for PD, taking as reference the smallest SLD since the treatment started.

According to the present invention the subject prior to treatment can comprise tumour and/or cancer cell AFP expression of greater than or equal to an intensity of 1+ in greater than or equal to 10, 15, 20, 25, 30%, preferably greater than or equal to 20% of tumour and/or cancer cells as determined by immunohistochemistry and non-cancerous AFP expression is less than or equal to 3, 5, 7, 9, 10% preferably less than or equal to 5% of cells for non-cancerous or non-tumour tissue at any intensity by immunohistochemistry.

According to the present invention the subject prior to treatment can comprise serum level AFP of greater than or equal to 50, 100, 200, 300 or 400 ng/mL preferably greater than or equal to 100 ng/ml and AFP expression is less than or equal to 3, 5, 7, 9, 10% preferably less than or equal to 5% of cells for non-cancerous or non-tumour tissue at any intensity by immunohistochemistry.

According to the present invention the subject prior to treatment may comprise an Eastern Cooperative Oncology Group (ECOG) of 0 to 1 and/or Child-Pugh score of any one of 1, 2, 3, 4, 5 or 6 and/or measurable disease according to Response Evaluation Criteria in Solid Tumors (RECIST) 1.1.

According to the present invention if prior to treatment the subject has any one or more of:
(a) serum AFP levels within the normal range, less than 100 ng/mL or less than or equal to ng/mL,
(b) liver transplant,
(c) immunotherapy with PD-1 or PD-L1 antagonist ligands and/or cytotoxic chemotherapy,
(d) HLA-C*04:04 positive or HLA-B*51:03 positive status, or
(e) loco-regional therapy; then
the subject is excluded from the treatment according to the invention.

According to the present invention the subject can be positive for HLA-A2, for example selected from HLA-A*02:01, HLA-A*02:02, HLA-A*02:03, HLA-A*02:04, HLA-A*02:05, HLA-A*02:06, HLA-A*02:642 or HLA-A*02:07, preferably HLA-A*02:01 or HLA-A*02:642 and/or the cancer and/or tumour expresses alpha fetoprotein (AFP), a peptide antigen of alpha fetoprotein (AFP), a peptide antigen of alpha fetoprotein (AFP) comprising FMNKFIYEI (SEQ ID No: 1) or residues 158-166 derived from alpha fetoprotein (AFP) SEQ ID NO: 51.

According to the invention the subject can be intolerant to a standard of care treatment, additionally or alternatively the subject and/or the cancer and/or tumour can have been previously unsuccessfully treated with a standard of care treatment, or been previously unsuccessfully treated with locoregional therapy optionally selected from chemical and/or thermal percutaneous ablation and intraarterial chemoembolotherapy. The standard of care treatment can be selected from any one of Sorafenib, a PD1 or PD-L1 antagonist or inhibitor, Regorafenib, Cabozantinib, Sunitinib, Brivanib, Everolimus, Tivantinib, Linifanib.

According to the present invention the cancer can be primary cancer, secondary cancer, relapsed cancer or refractory cancer or recurrent cancer or locally recurrent cancer or metastatic cancer, non-resectable cancer or locally confined, cancer with no surgical or radiotherapy option or inoperable cancer, cancer which is not amenable to transplant or loco-regional therapy or any combination thereof. The subject may have relapsed cancer or refractory cancer or recurrent cancer or locally recurrent cancer or metastatic cancer or locally confined or inoperable cancer, or any combination thereof.

According to the present invention the cancer may be selected from; lung cancer, non-small cell lung cancer (NSCLC), metastatic or advanced NSCLC, squamous NSCLC, adenocarcinoma NSCLC, adenosquamous NSCLC, large cell NSCLC, ovarian cancer, gastric cancer, urothelial cancer, esophageal cancer, esophagogastric junction cancer (EGJ), melanoma, bladder cancer, head and neck cancer, head and neck squamous cell carcinoma (HNSCC), cancer of the oral cavity, cancer of the oropharynx, cancer of the hypopharynx, cancer of the throat, cancer of the larynx, cancer of the tonsil, cancer of the tongue, cancer of the soft palate, cancer of the pharynx, synovial sarcoma, myxoid round cell liposarcoma (MRCLS), optionally wherein the cancer or tumour express a AFP or peptide antigen thereof, optionally a peptide antigen of alpha fetoprotein (AFP) comprising FMNKFIYEI (SEQ ID No: 1) or residues 158-166 derived from alpha fetoprotein (AFP) SEQ ID NO: 51.

According to the present invention the cancer may be selected from any one of breast cancer, metastatic breast cancer, liver cancer, renal cell carcinoma, synovial sarcoma, urothelial cancer or tumour, pancreatic cancer, colorectal cancer, metastatic stomach cancer, metastatic gastric cancer, metastatic liver cancer, metastatic ovarian cancer, metastatic pancreatic cancer, metastatic colorectal cancer, metastatic lung cancer, colorectal carcinoma or adenocarcinoma, lung carcinoma or adenocarcinoma, pancreatic carcinoma or adenocarcinoma, mucinous adenoma, ductal carcinoma of the pancreas, hematological malignancy, optionally wherein the cancer or tumour express a AFP or peptide antigen thereof, optionally a peptide antigen of alpha fetoprotein (AFP) comprising FMNKFIYEI (SEQ ID No: 1) or residues 158-166 derived from alpha fetoprotein (AFP) SEQ ID NO: 51.

According to the invention the cancer can be liver cancer, or can be liver cancer selected from any of; cholangiocarcinoma, liver angiosarcoma, hepatoblastoma, hepatocellular carcinoma (HCC), optionally wherein the cancer is not amenable to transplant or resection, preferably the cancer is hepatocellular carcinoma (HCC). Additionally the liver cancer may be coincident with any one or more of; diabetes, obesity, hepatitis B, hepatitis C, cirrhosis.

There is further provided the treatment method or use according to the invention wherein the subject has not received prior treatment for cancer and/or tumour, alternatively wherein the subject has received prior treatment for cancer and/or tumour and/or has failed to respond to prior cancer treatment for cancer and/or tumour.

According to the invention the prior treatment can comprise systemic and/or local therapy, for example any one or more of; surgery, radiation therapy, cryotherapy, laser therapy, topical therapy, chemotherapy, hormonal therapy, targeted drugs, or immunotherapy. Accordingly, the prior treatment can comprise local therapy, for example any one or more of surgery, radiation therapy cryotherapy, laser therapy, topical therapy and/or systemic therapy, for example any one or more of chemotherapy, hormonal therapy, targeted drugs, or immunotherapy.

According to the invention the prior treatment can comprise a PD-1 axis binding antagonist, PD-L1 binding antagonist or PD-1 binding antagonist. Accordingly the prior treatment can comprise any of;
(a) an anti-PD-L1 antibody which inhibits binding between PD-L1 and PD-1 and/or between PD-L1 and B7-1,
(b) an anti-PD-L1 antibody which inhibits PD-L1 on the cancer cell surface from transducing a signal to the intracellular pathway,
(c) an anti-PD-1 antibody which inhibits binding between PD-L1 and PD-1 and/or between PD-L2 and PD-1,
(d) an anti-PD-1 antibody which inhibits PD-1 on the T cell surface from transducing a signal to the intracellular pathway,
(e) a PD-L1 binding antagonist which is selected from;
   (i) Durvalumab, Imfinzi or MED14736,
   (ii) Atezolizumab, Tecentriq or MPDL3280A,
   (iii) Avelumab, Bavencio or MSB0010718C,
   (iv) MDX-1105, BMS-936559,
(f) a PD-1 binding antagonist is selected from;
   (i) Pembrolizumab, Keytruda, Lambrolizumab or MK-3475,
   (ii) Cemiplimab, Libtayo, or REGN-2810,
   (iii) BMS/ONO, Nivolumab, Opdivo, ONO-4538, BMS-936558 or MDX1106.

According to the invention the prior treatment may comprise an Epidermal Growth Factor Receptor Antagonist, optionally Cetuximab. According to the invention when the prior treatment comprises chemotherapy this may comprise one or more platinum compound, optionally selected from Lipoplatin, Cisplatin, Carboplatin, Oxaliplatin, Nedaplatin, Triplatin tetranitrate, Phenanthriplatin, Satraplatin, Picoplatin. Additionally or alternatively when the prior treatment comprises chemotherapy this may comprise one or more chemotherapeutic agent selected from, methotrexate, capecitabine, taxane, anthracycline, paclitaxel, docetaxel, paclitaxel protein bound particles, doxorubicine, epirubicine, 5-fluorouracil, cyclophosphamide, afatinib, vincristine, etoposide or combinations thereof. Additionally, or alternatively when the prior treatment comprises chemotherapy this may comprise one or more chemotherapeutic agent selected from, FEC: 5-fluorouracil, epirubicine, cyclophosphamide; FAC: 5-fluorouracil, doxorubicine, cyclophosphamide; AC: doxorubicine, cyclophosphamide; EC: epirubicine, cyclophosphamide.

According to the invention the prior treatment can comprise any one or more of Sorafenib, a PD1 or PD-L1 antagonist or inhibitor, Regorafenib, Cabozantinib, Sunitinib, Brivanib, Everolimus, Tivantinib, Linifanib, or locoregional therapy optionally selected from chemical and/or thermal percutaneous ablation and intraarterial chemoembolotherapy.

According to the invention the subject may not have received prior treatment in recurrence less than or equal to 12 months since the last treatment or less than or equal to 6 months since the last treatment. According to the invention the subject may have not received any prior adjuvant therapy (surgery followed by radiation and/or chemotherapy) in recurrence less than or equal to 12 months since the last treatment or in recurrence less than or equal to 6 months since the last treatment.

According to the invention the treatment extends or improves or effectively extends or effectively improves any of:
(a) progression free survival,
(b) time to progression,
(c) duration of response,
(d) overall survival,
(e) objective response or objective response rate,
(f) overall response or overall response rate,
(g) partial response or partial response rate,
(h) complete response or complete response rate;
(i) stable disease rate or median stable disease
(j) median progression free survival,
(k) median time to progression,
(l) median duration of response, or
(m) median overall survival;
(n) median objective response or median objective response rate,
(o) median overall response or median overall response rate,
(p) median partial response or median partial response rate,
(q) median complete response or median complete response,
(r) median stable disease rate or median stable disease,
in comparison to placebo treatment or in comparison to prior to treatment or in comparison to without treatment or in comparison to treatment comprising a standard of care, optionally wherein the standard of care treatment is selected from any one of Sorafenib, a PD1 or PD-L1 antagonist or inhibitor, Regorafenib, Cabozantinib, Sunitinib, Brivanib, Everolimus, Tivantinib, Linifanib.

“Progression free survival” (PFS) refers to the time from treatment (or randomization) to first disease progression or death. “Time to progression” (TTP) does not count patients who die from causes other than the cancer or tumour being treated but is otherwise equivalent to PFS. “Duration of response” (DoR), is the length of time that cancer, tumour or lesion continues to respond to treatment without growing or spreading. According to the invention DoR, TTP and PFS can be assessed by Response Evaluation Criteria in Solid Tumors (RECIST) or can be assessed by CA-125 levels (cancer antigen 125) as a determinant of progression.

According to the invention PFS and/or TTP and/or DoR, or median thereof, can be extended or improved by at least 1 month, 2 months, 2.3 months, 2.5 months, 2.9 months, 3 months, 3.5 months, 3.8 months, 4 months, 4.5 months, 5 months, 6 months, 7 months, 8 months, 9 months, 10 months, 11 months, 12 months, 16 months, 18 months, 20 months, 22 months, 2 years, 3 years, 4 years, 5, years, 6 years, 7 years, 8 years, 9 years, or 10 years in comparison to placebo treatment or in comparison to prior to treatment or in comparison to without treatment or in comparison to treatment comprising a standard of care for example as herein described (a “control”).

In one embodiment, the PFS and/or TTP and/or DoR, or median thereof, is extended about 2.9 months to 3.8 months compared to the control. In one embodiment, the PFS and/or TTP and/or DoR, or medians thereof, is extended at least about 3.8 months compared to the control. In another embodiment, the PFS and/or TTP and/or DoR, or median thereof, is extended by about 2.3 months, in one embodiment, the PFS and/or TTP and/or DoR, or median thereof, is extended about 6 months compared to a “control”.

“Overall survival” refers to a subject remaining alive for a defined period of time. According to the invention the overall survival, or median thereof, is improved or extended by about 6 months, about 1 year, about 1.5 years, about 2 years, about 3 years, about 4 years, about 5 years, about 6 years, about 7 years, about 8 years, about 9 years, about 10 years, from initiation of the method or treatment according to the invention or from initial diagnosis, optionally the event used for survival analysis can be death from any cause. “Survival” refers to a subject remaining alive and includes progression free survival (PFS) and overall survival (OS). “Overall survival” is the length of time from either the date of diagnosis or the start of treatment for the disease, tumour and/or cancer, that subjects diagnosed with the disease are still alive. Survival can be estimated by the Kaplan-Meier method, and any differences in survival are computed using the stratified log-rank test; “extending survival” or “increasing the likelihood of survival” is meant increasing PFS and/or OS in a treated subject in comparison to placebo treatment or in comparison to prior to treatment or in comparison to without treatment or in comparison to treatment comprising a standard of care (a “control”). According to the invention overall survival or survival can be extended or improved by at least 1 month, 2 months, 2.3 months, 2.5 months, 2.9 months, 3 months, 3.5 months, 3.8 months, 4 months, 4.5 months, 5 months, 6 months, 7 months, 8 months, 9 months, 10 months, 11 months, 12 months, 16 months, 18 months, 20 months, 22 months, 2 years, 3 years, 4 years, 5, years, 6 years, 7 years, 8 years, 9 years, 10 years in comparison to placebo treatment or in comparison to prior to treatment or in comparison to without treatment or in comparison to treatment comprising a standard of care (a “control”).

“Objective response rate” (ObRR) is the proportion of subjects with tumour size reduction of a predefined amount, optionally determined by sum of the longest diameters (SLD) of target lesions or tumours, and for a minimum time period. “Overall response rate (ORR)” is defined as the proportion of subjects who have a partial or complete response to therapy; it does not include stable disease. ORR is generally defined as the sum of complete responses (CR) and partial responses (PRs) over a specified time period. According to the invention ObRR and/or ORR and/or PR and/or CR and/or SD can be extended or improved by at least 5%, 10%, 15%, 20%, 25%, 30%, 35%, 40%, 45%, 50%, 55%, 60%, 65%, 70%, 75%, 80%, 85%, 90%, 95%, in comparison to placebo treatment or in comparison to prior to treatment or in comparison to without treatment or in comparison to treatment comprising a standard of care (a “control”).

According to the present invention, the method may further comprise determining the expression level of a biomarker in a sample from the subject wherein the level of the biomarker is compared to a reference level in order to determine the subject's likelihood to respond to the treatment or to determine the subject's level of response to the treatment, wherein the sample is obtained either before during or after the treatment. The reference level may be the level prior to treatment of the subject or may be the level associated with the presence of cancer or the lack of presence of cancer. The biomarker may be a T-effector-associated gene, for example CD8A, perforin (PRF1), granzyme A (GZMA), granzyme B (GZMB), interferon-γ (IFN-v), CXCL9, or CXCL10. The biomarker may be an activated stroma-associated gene, for example transforming growth factor-β (TGF-β), fibroblast-activated protein (FAP), podplanin (PDPN), a collagen gene, or biglycan (BGN). The biomarker may be a or a myelokJ-derived suppressor cell-associated gene, for example CD68, CD163, FOXP3, or androgen-regulated gene 1. Alternatively the biomarker may be PD-L1, CD8, or androgen receptor (AR) gene.

According to the present invention the subject undergoes lymphodepleting chemotherapy prior to administration of the modified immunoresponsive cells expressing or presenting a heterologous T-cell receptor (TCR). The lymphodepleting chemotherapy may comprise administration of cyclophosphamide and/or fludarabine. Preferably the cyclophosphamide is administered at a dose of about 100, 150, 200, 250, 300, 350, 400, 450, 500, 550, 600, 650, 700, 750, 800 or 850 mg/m²/d [d=day], preferably about 500 or 600 mg/m²/d, preferably wherein the administration is for 1 day, 2 days (×2d), 3 days (×3d), 4 days (×4d) or 5 days (×5d). Preferably the fludarabine is administered at a dose of about 5, 10, 15, 20, 25, 30, 35, 40, 450, 50, 55, 60, 65, 70, 75, 80 or 85 mg/m²/d, preferably wherein the administration is for 1 day, 2 days (×2d), 3 days (×3d), 4 days (×4d) or 5 days (×5d). Preferably the lymphodepleting chemotherapy comprises administration of cyclophosphamide and fludarabine optionally at a dose of 500 mg/m²/d×3d cyclophosphamide and 20 mg/m2/d×3d fludarabine or at a dose of 600 mg/m²/d×3d cyclophosphamide and 30 mg/m2/d×4d. According to the invention the lymphodepleting chemotherapy can administered at 3, 4, 5, 6, 7, 8, 9, 10 days preferably 7 to 5 or 7 to 4 days prior to administration of the modified immunoresponsive cells expressing or presenting a heterologous T-cell receptor (TCR). The administration of cyclophosphamide and fludarabine may be sequential separate or simultaneous, the administration may be administered intravenously or by intravenous infusion.

The invention further provides a method of
(a) reducing serum AFP expression or concentration,
(b) enhancing immune function,
(c) reducing tumour growth or tumour growth rate or maintaining tumour size after cessation of treatment or reducing tumour number or tumour burden,
(d) increasing serum cytokine and/or interferon level or concentration
(e) improving T-cell persistence, or
(f) improving T-cell infiltration of tumour,
   in a subject having cancer and/or tumour comprising administering to the subject treatment regimen comprising an effective amount of modified immunoresponsive cells expressing or presenting a heterologous T-cell receptor (TCR) which binds AFP or a peptide antigen of AFP or a peptide antigen of AFP comprising FMNKFIYEI (SEQ ID No: 1) or residues 158-166 derived from alpha fetoprotein (AFP) SEQ ID NO: 51, as herein before described with reference to the method of treatment and the aspects and embodiments and features relating thereto, optionally in comparison to prior to treatment or without treatment or treatment comprising a standard of care as herein described. The standard of care treatment may be selected from any one of Sorafenib, a PD1 or PD-L1 antagonist or inhibitor, Regorafenib, Cabozantinib, Sunitinib, Brivanib, Everolimus, Tivantinib, Linifanib.

Accordingly, the invention provides a method of enhancing immune function wherein:
(a) CD8 T cells in the individual have enhanced priming, activation, proliferation and/or cytolytic activity,
(b) the number of CD8 T cells is elevated,
(c) the cancer and/or tumour cells in the subject selectively have elevated expression of MHC class I antigen expression, optionally wherein PBMC cells of the subject do not have elevated expression of MHC class I antigen,
(d) the antigen presenting cells in the subject have enhanced maturation and activation, optionally wherein the antigen presenting cells are dendritic cells,
(e) the serum levels of IL-10 and/or IL-8 in the individual are reduced,
(f) the cancer and/or tumour of the subject has elevated levels of T-cell infiltration, or
(g) the T cells of the subject have reduced levels of T-cell PD-1 expression; respectively and optionally in comparison to prior to treatment or without treatment or treatment comprising a standard of care as hereinabove described.

Accordingly (a) the CD8 T cell activation may be characterised by an elevated frequency of gamma-IFN⁺ CD8 T cells and/or enhanced cytolytic; (b) the maturation of the antigen presenting cells may be characterised by increased frequency of CD83⁺ dendritic cells; (c) the activation of the antigen presenting cells may be characterised by elevated expression of CD80 and CD86 on dendritic cells; or (d) the CD8 T cell may be an antigen-specific CD8 T cell.

According to the invention there is provided;
(a) a kit comprising an effective amount of modified immunoresponsive cells expressing or presenting a heterologous T-cell receptor (TCR) which binds AFP or a peptide antigen of AFP or a peptide antigen of AFP comprising FMNKFIYEI (SEQ ID No: 1) or residues 158-166 derived from alpha fetoprotein (AFP) SEQ ID NO: 51 and a package insert comprising instructions for using the modified immunoresponsive cells to treat or delay the progression of cancer and/or tumour in a subject,
(b) a kit comprising an effective amount of modified immunoresponsive cells expressing or presenting a heterologous T-cell receptor (TCR) which binds AFP or a peptide antigen of AFP or a peptide antigen of AFP comprising FMNKFIYEI (SEQ ID No: 1) or residues 158-166 derived from alpha fetoprotein (AFP) SEQ ID NO: 51 and a package insert comprising instructions for using the modified immunoresponsive cells in a method of
   (i) reducing serum AFP expression or concentration,
   (ii) enhancing immune function,
   (iii) reducing tumour growth or tumour growth rate or maintaining tumour size after cessation of treatment or reducing tumour number or tumour burden,
   (iv) increasing serum cytokine and/or interferon level or concentration
   (v) improving T-cell persistence, or
   (vi) improving T-cell infiltration of tumour,
      in a subject having cancer and/or tumour as herein before described.

The invention will be further described by reference to the following figures and examples.

### DESCRIPTION OF FIGURES

FIG. 1. (SEQ ID No: 2) gives the amino acid sequence of the extracellular part of the alpha chain of the parental AFP-specific TCR with gene usage TRAV12-2*02/TRAJ41*01/TRAC.

FIG. 2. (SEQ ID No: 3) gives the amino acid sequence of the extracellular part of the beta chain of the parental AFP-specific TCR with gene usage TRBV9*01/TRBD2/TRBJ2-7*01/TRBC2.

FIG. 3. (SEQ ID No: 4) gives the amino acid sequence of the alpha chain of a soluble TCR (referred to herein as the “reference TCR”). The sequence is the same as that of FIG. 1 (SEQ ID No: 2) except that a cysteine (bold and underlined) is substituted for T159 of SEQ ID No: 2 (i.e. T48 of the TRAC constant region).

FIG. 4. (SEQ ID No: 5) gives the amino acid sequence of the beta chain of a soluble TCR (referred to herein as the “reference TCR”). The sequence is the same as that of FIG. 2 (SEQ ID No: 3) except that a cysteine (bold and underlined) is substituted for S169 of SEQ ID No: 3 (i.e. S57 of the TRBC2 constant region) and A187 is substituted for C187 and D201 is substituted for N201.

FIG. 5. (SEQ ID Nos: 6-20) gives the amino acid sequence of the mutated alpha chains which may be present in TCRs of the invention. The CDR regions are underlined and amino acid changes relative to the parental AFP TCR are shaded.

FIGS. 6. (SEQ ID No: 21) and (SEQ ID No: 22) gives the DNA sequences encoding the TCR alpha and beta chains shown in FIGS. 3 and 4 respectively

FIG. 7. (SEQ ID No: 23) gives the DNA sequence for the parental AFP TCR gene (alpha chain-2A-beta chain construct with the Porcine teschovirus-1 2A sequence bold and underlined) for transduction of T-cells.

FIG. 8. (SEQ ID No: 24) gives the amino acid sequence of the parental AFP TCR for T-cell transduction produced from the DNA sequence of FIG. 7. The Porcine teschovirus-1 2A sequence is bold and underlined.

FIG. 9. (SEQ ID NO:25-43) shows the DNA sequences of cloning primers for production of AFP TCRs and amino acid sequences of variant TCR alpha chains.

FIG. 10. (SEQ ID NO:44-48) shows the amino acid and DNA sequences of CD8.

FIG. 11. (SEQ ID NO: 49 and 50) shows AFP TCR variant alpha and beta chain amino acid sequences.

FIG. 12. (SEQ ID NO: 51) Human Alpha-fetoprotein amino acid sequence

FIG. 13. ADP-A2AFP (AFPc332T) engineered T-cell treatment, 5 Billion cell dose target, (Cy: 600 mg/m²/d)×3d; (Flu: 30 mg/m²/d)×4d, CT scan of subject liver immediately prior to treatment (Baseline) and 8 weeks after commencement of treatment showing complete response, 100% decrease in target lesions (indicated by arrows).

FIG. 14. ADP-A2AFP (AFPc332T) engineered T-cell treatment, 5 Billion cell dose target, (Cy: 600 mg/m²/d)×3d; (Flu: 30 mg/m²/d)×4d, serum AFP level over 8 week period following commencement of treatment.

FIG. 15. ADP-A2AFP (AFPc332T) engineered T-cell treatment, 5 Billion cell dose target, (Cy: 600 mg/m²/d)×3d; (Flu: 30 mg/m²/d)×4d, serum level persistence of AFPc332T engineered T-cell as measured by AFPc332T vector copy number.

FIG. 16. ADP-A2AFP (AFPc332T) engineered T-cell treatment, 5 Billion cell dose target, (Cy: 600 mg/m²/d)×3d; (Flu: 30 mg/m²/d)×4d, serum level persistence of AFPc332T engineered T-cell as measured by AFPc332T transduced T-cell number.

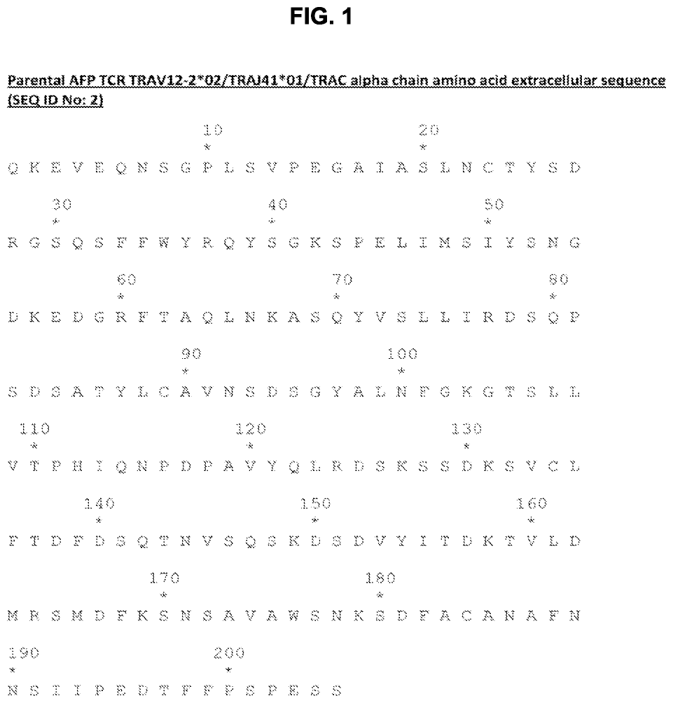

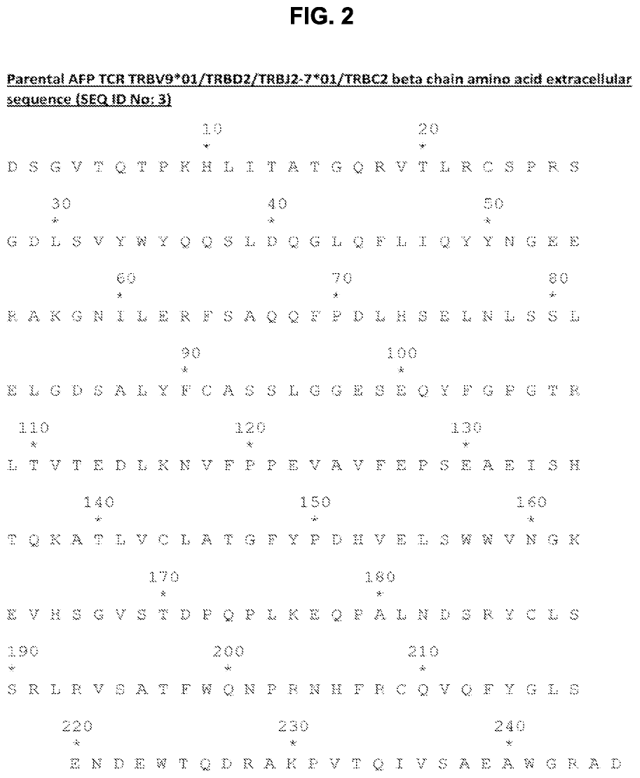

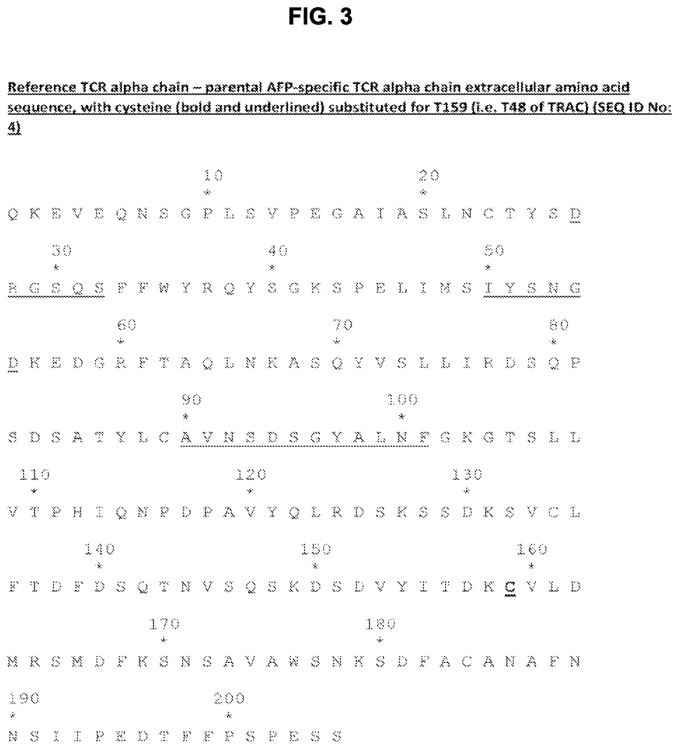

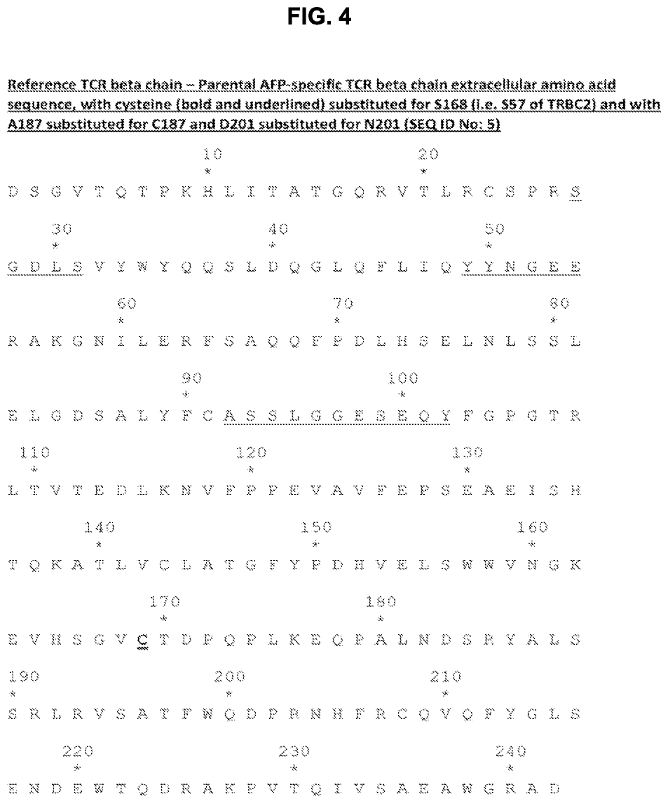

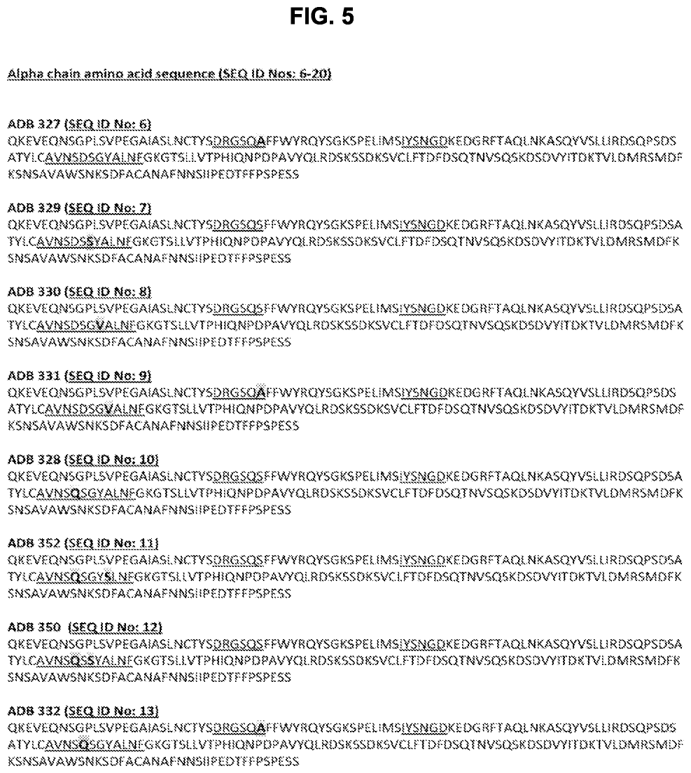

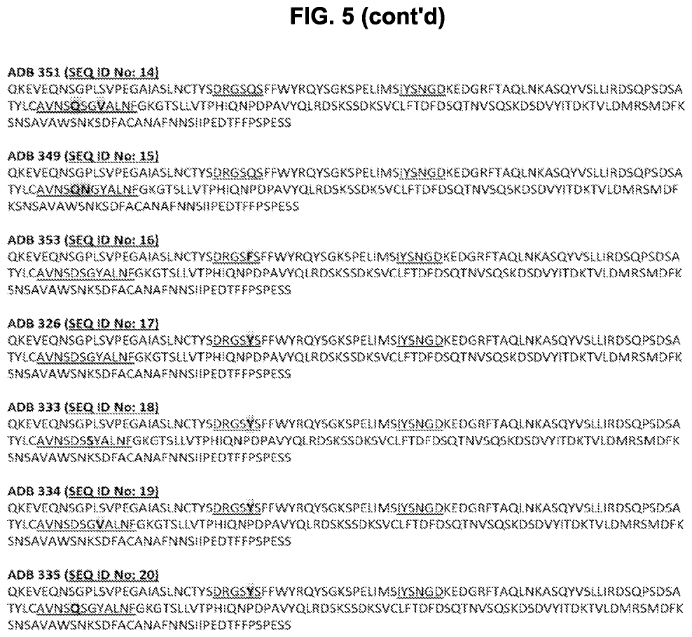

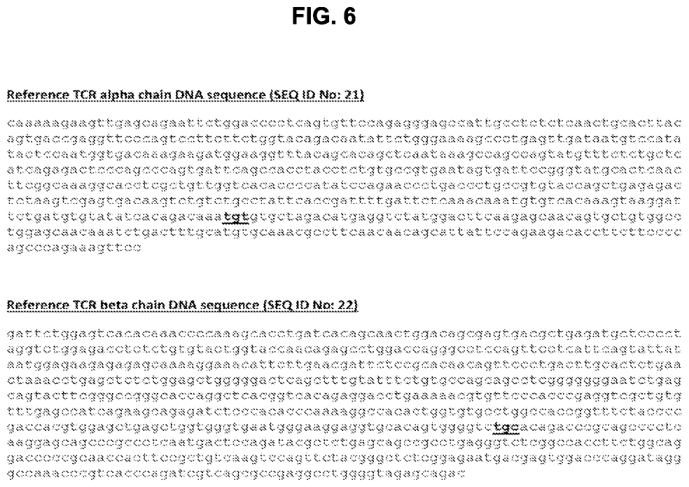

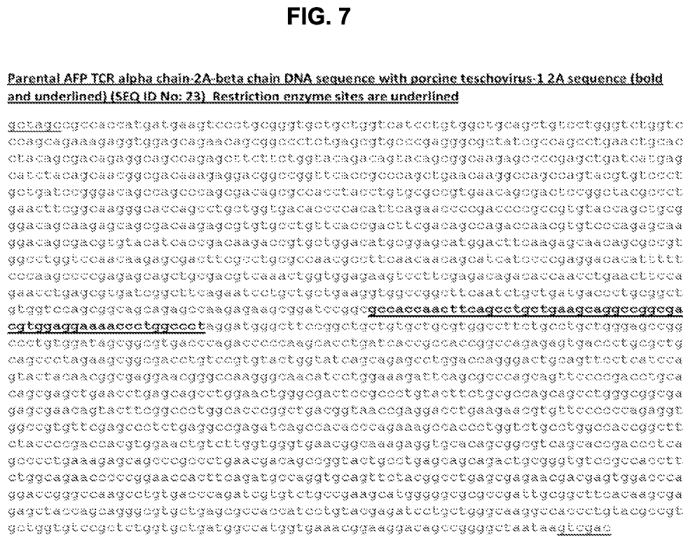

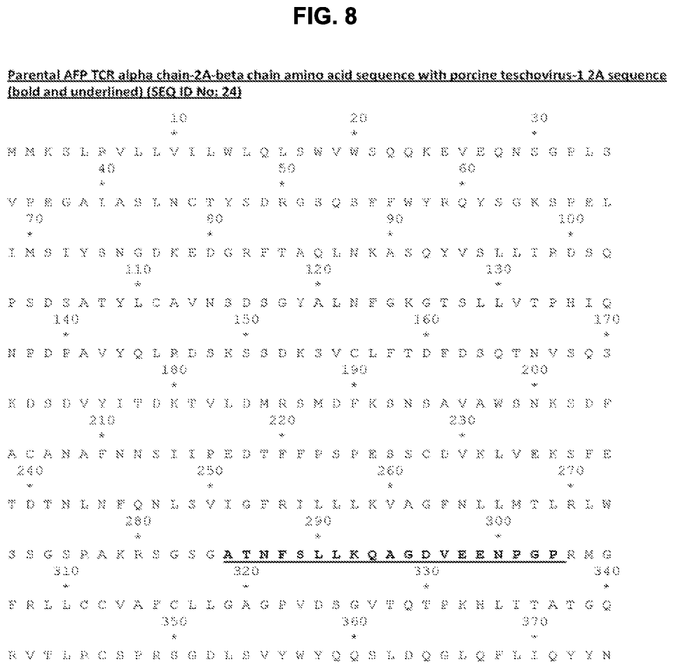

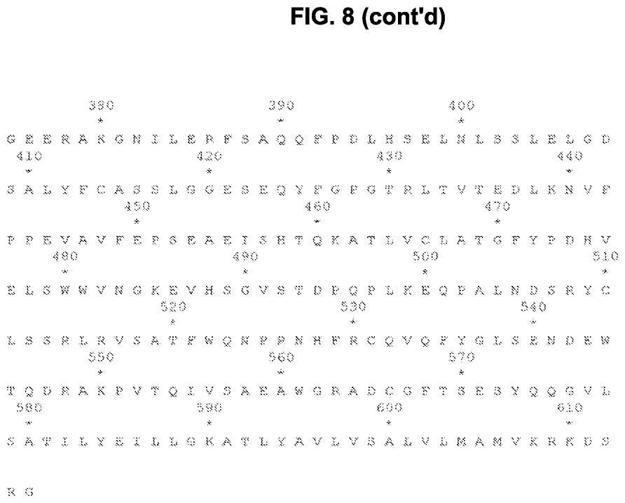

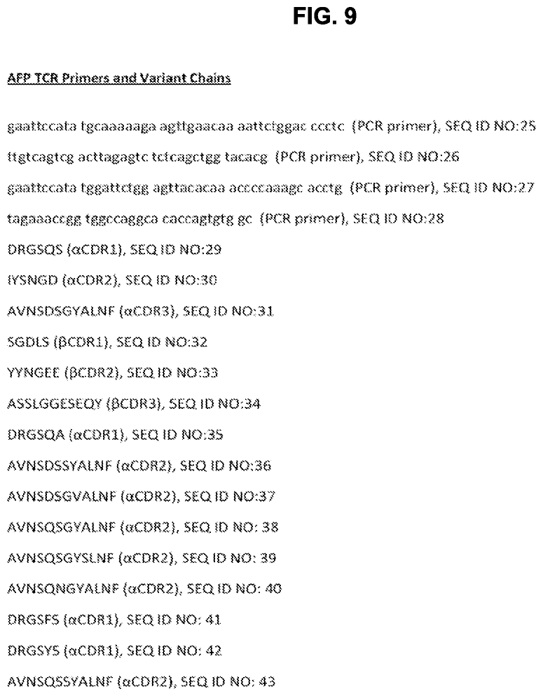

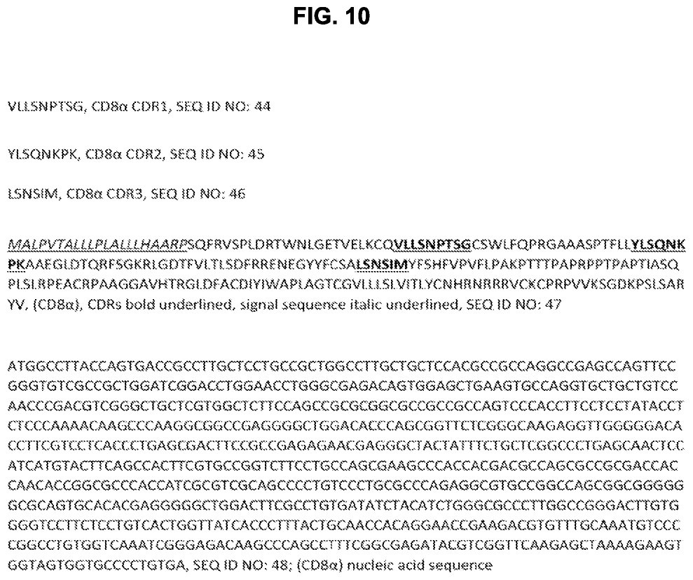

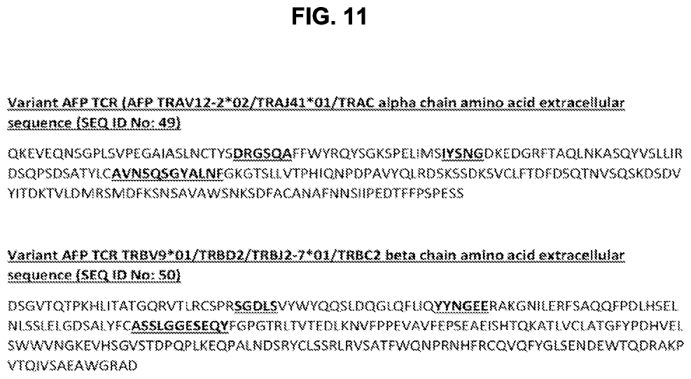

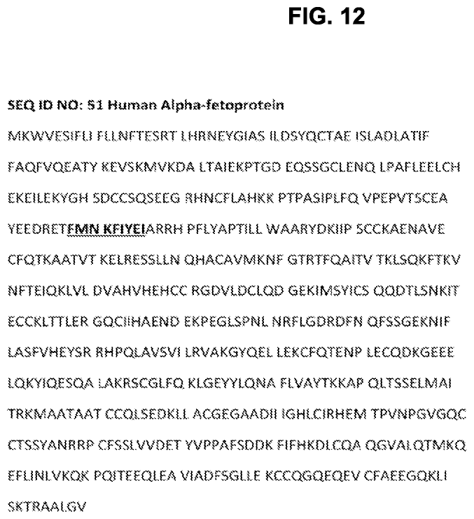

The invention is further described in the following non-limiting examples.

### EXAMPLES

### Example 1—A Phase I Open Label, Clinical Trial Evaluating the Safety and Anti-Tumour Activity of Autologous T Cells Expressing Enhanced TCRs Specific for Alpha-Fetoprotein (AFPc332T) in HLA-A2 Positive Subjects with Advanced Hepatocellular Carcinoma (HCC) or Other AFP Expressing Tumour Types

### Methods

The following presents an in human study of genetically engineered AFPc332T cells in HLA-A *02:01 P group positive subjects with advanced HCC or other AFP expressing tumour types. Disease may be histologically or cytogenetically confirmed and/or measurable disease according to RECIST v1.1. Subjects who are eligible based on HLA type and who met AFP criteria were screened for general health, performance status and disease stage. Subjects must have a relative absence of AFP expression in their non-cancerous liver tissue. Following Screening, subjects meeting all eligibility criteria underwent leukapheresis to obtain cells for the manufacture of autologous AFP TCR bearing T-cells. The cells are subsequently transduced with the ADP-A2AFP, AFPc332T TCR (SEQ ID NO: 49, 50) specific for AFP antigen (particularly the specific AFP antigenic peptide SEQ ID NO:1) and the cells expanded and cryopreserved for later use. Once the AFPc332T cells are available, subjects undergo lymphodepleting chemotherapy with cyclophosphamide plus fludarabine on Days −7 to −5, or Days −7 to −4 followed by infusion of transduced cells on Day 1.

Three subject cohorts were treated dosing with between 100 million to 5 billion transduced cells respectively with no dose escalation:
100 mn cell dose, (cyclophosphamide: 500 mg/m²/d)×3d; (fludarabine: 20 mg/m²/d)×3d
1 bn cell dose, (cyclophosphamide: 500 mg/m²/d)×3d; (fludarabine: 20 mg/m²/d)×3d
5 bn cell dose, (cyclophosphamide: 600 mg/m²/d)×3d; (fludarabine: 30 mg/m²/d)×4d

Subjects are hospitalised for 7 days following infusion and monitored for safety, T-cell persistence, cytokine production with CT and MRI performed at weeks 4, 8, 16, 24 and 3 monthly thereafter until disease progression or early interventional withdrawal, long term follow up annually is planned for a 15 year period.

A subject will be considered completing the interventional phase of the study when he/she has received T-cell infusion and then progressed or died prior to disease progression. Optionally a second T-cell infusion may be given, and they will remain in the interventional phase of the study until they have further progression of disease. Once progression is established, no further efficacy assessments are performed other than overall survival. All subjects completing from the interventional portion of the study will enter the long-term follow-up (LTFU) phase for observation of delayed adverse events (AEs) during the 15 years post-infusion in accordance with FDA and EMA regulations. This study will be considered complete when the last living subject has completed LTFU. The study covers treatment of AFP expressing tumours including hepatocellular carcinoma and other AFP expressing tumours.

To evaluate the safety and tolerability of AFPc332T the incidence of dose limiting toxicities (DLTs) is monitored, determination is made of optimally tolerated dose range, adverse events (AEs), and Serious Adverse Events (SAEs); laboratory assessments, including chemistry, haematology, and coagulation; and cardiac assessments, including ECG and cardiac Troponin.

During the study serum AFP is evaluated as the biomarker for tumour AFP expression, and antitumor activity. This is performed to correlate the level of antigen expression in tumour and serum AFP level at Baseline, and post AFPc332T cell infusion. Correlation of changes in serum AFP from baseline with response to treatment is thereby assessed. Post-therapy AFP expression in tumour over time is assessed to determine tumour immunity or resistance to AFPc332T. Additionally, circulating cytokines were measured and evaluated for association with cytokine release syndrome (CRS) and other adverse events (AEs). Additionally post AFPc332T cell infusion, transduced cell persistence is assessed by determination of serum level persistence of AFPc332T engineered T-cell as measured by AFPc332T vector copy number and AFPc332T transduced T-cell number.

Key inclusion criteria for subjects includes:
1. Histologically confirmed AFP expressing HCC or histologically confirmed diagnosis of another AFP expressing tumour, not amenable to transplant or resection.
2. Measurable disease according to Response Evaluation Criteria in Solid Tumours (RECIST) 1.1 criteria prior to lymphodepletion.
3. Progressive disease following or intolerant of or refuses standard of care systemic therapy prior to lymphodepletion.
4. Positive for HLA-A*02:01 (or any A*02:01 P group allele).
5. AFP expression of ≥1+ in ≥20% of tumour cells by immunohistochemistry and their non-cancerous liver tissue has ≤5% cells stained for AFP at any intensity by immunohistochemistry.
6. Serum AFP levels of 100 ng/mL and their non-cancerous liver tissue has ≤5% cells stained for AFP at any intensity by immunohistochemistry.
7. Life expectancy of >4 months
8. Child-Pugh score≤6
9. Eastern Cooperative Oncology Group (ECOG) 0-1
10. Female subjects of childbearing potential (FCBP) must have a negative serum pregnancy test.

Key exclusion criteria for subjects included: (a) positive for the following alleles: HLA-C*04:04 or HLA-B*51:03, (b) Prior liver transplant, (c) received the following prior to leukapheresis: i) Cytotoxic chemotherapy, immune therapy and biological therapy within 3 weeks, ii) Corticosteroids or any other immunosuppressive therapy within 2 weeks.

To evaluate anti-tumour activity of AFPc332T the following endpoints are monitored by RECIST v1.1; Overall Response Rate (ORR) defined as the proportion of subjects with a confirmed complete response (CR) or partial response (PR). Additional endpoints are monitored for duration of response (DoR), duration of stable disease (SD), progression free survival (PFS), overall survival (OS).

### Results

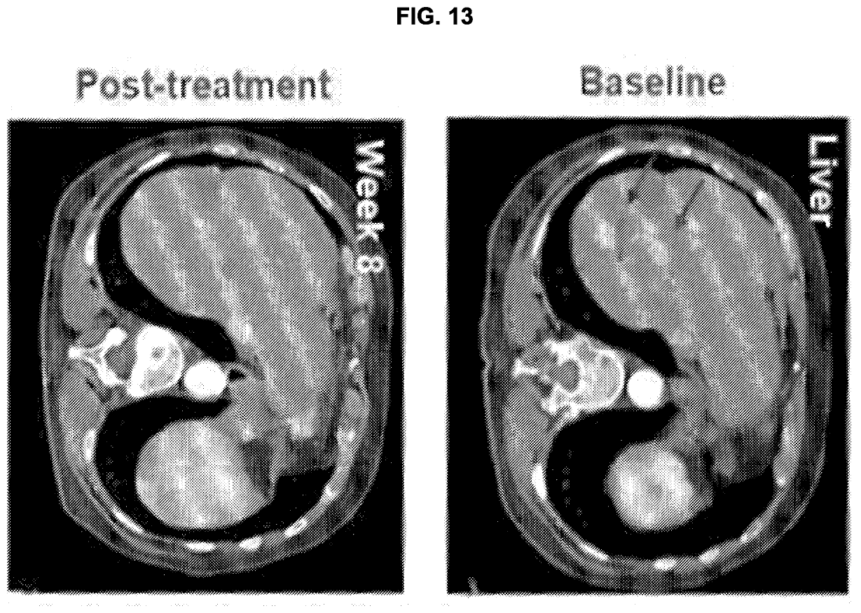

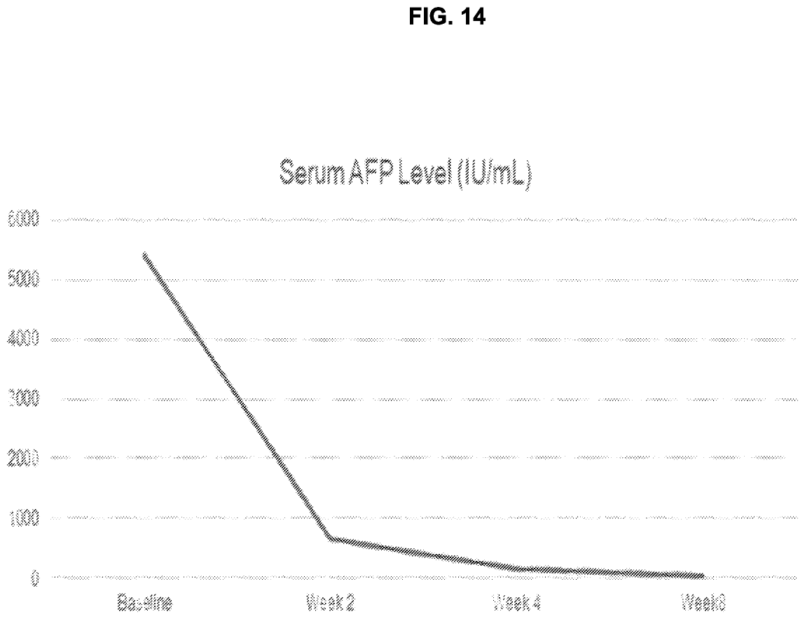

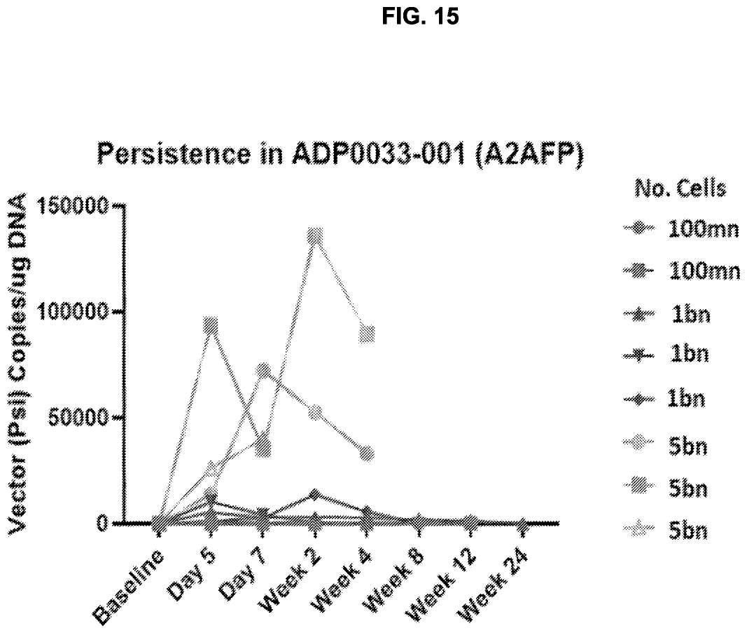

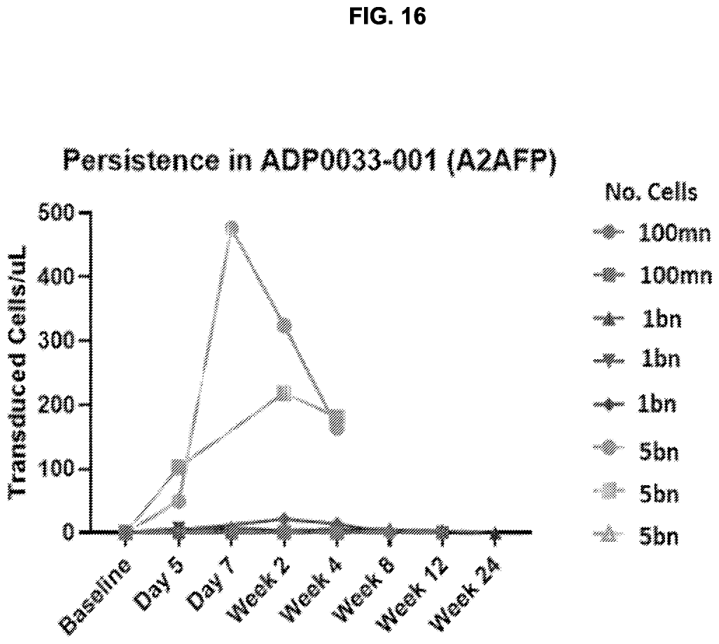

Data shown in FIG. 13 demonstrates a 100% decrease in target lesions at an 8 week period following AFPc332 T-cell infusion for the cohort receiving a 5 billion cell infusion, FIG. 14 shows that this is coincident with a sharp decrease of serum AFP levels indicative of a measurable response to treatment and of tumour AFP expression, this biomarker change indicates treatment efficacy of cancer and tumour treatment for HCC in the cohort subjects. AFPc332 T-cell serum vector and transduced T-cell measures up to 8 weeks from infusion indicate that the engineered AFPc332 T-cells persist well in the subject retain the engineered TCR for the HCC cohort subjects dosed at 5 billion cells.

1. A method of treating, preventing or delaying the progression of cancer and/or tumour in a subject comprising administering to the subject a treatment regimen comprising an effective amount of modified immunoresponsive cells expressing or presenting a heterologous T-cell receptor (TCR) which binds a peptide antigen of alpha fetoprotein (AFP) comprising FMNKFIYEI (SEQ ID No: 1) or residues 158-166 derived from alpha fetoprotein (AFP) SEQ ID NO: 51.

2. The method according to claim 1, wherein:
(a) the heterologous TCR binds specifically and/or selectively to the peptide antigen, optionally wherein the peptide antigen is associated with a cancerous condition, cancer and/or tumour and/or is presented by tumour of cancer cell or tissue;
(b) the cancer and/or tumour is an AFP expressing cancer and/or tumour, and/or expresses alpha fetoprotein or peptide antigen thereof or a peptide antigen of alpha fetoprotein (AFP) comprising FMNKFIYEI (SEQ ID No: 1) or residues 158-166 derived from alpha fetoprotein (AFP) SEQ ID NO: 51; or
(c) the peptide antigen is complexed with a peptide presenting molecule, optionally wherein the peptide presenting molecule is (i) major histocompatibility complex (MHC), (ii) human leukocyte antigen (HLA), optionally class I or class II, or (iii) HLA-A*02, optionally selected from HLA-A*02:01, HLA-A*02:02, HLA-A*02:03, HLA-A*02:04, HLA-A*02:05, HLA-A*02:06, HLA-A*02:642 or HLA-A*02:07, preferably HLA-A*02:01 or HLA-A*02:642.

3-6. (canceled)

7. The method of claim 1, wherein the heterologous TCR binds specifically and/or selectively to the peptide antigen and/or the peptide presenting molecule and/or complex thereof.

8. The method according to claim 1, wherein the heterologous TCR comprises:
(a) a TCR alpha chain variable domain and a TCR beta chain variable domain, wherein:
(i) the alpha chain variable domain comprises CDRs having the sequences
DRGSQS (αCDR1), SEQ ID NO:29 or amino acids 27-32 of SEQ ID NO:2, or sequence having at least 50% sequence identity thereto,
IYSNGD (αCDR2), SEQ ID NO:30 or amino acids 50-55 of SEQ ID NO:2, or sequence having at least 50% sequence identity thereto, and
AVNSDSGYALNF (αCDR3), SEQ ID NO:31 or amino acids 90-101 of SEQ ID NO:2, or sequence having at least 50% sequence identity thereto, and
(ii) the beta chain variable domain comprises CDRs having the sequences
SGDLS (βCDR1), SEQ ID NO:32 or amino acids 27-31 of SEQ ID NO:3, or sequence having at least 50% sequence identity thereto,
YYNGEE (βCDR2), SEQ ID NO:33 or amino acids 49-54 of SEQ ID NO:3, or sequence having at least 50% sequence identity thereto, and
ASSLGGESEQY (βCDR3), SEQ ID NO:34 or amino acids 92-102 of SEQ ID NO:3;
or sequences having at least 50% sequence identity thereto; or
(b) a TCR in which the alpha chain variable domain comprises an amino acid sequence that has at least 80%, identity to the sequence of amino acid residues 1-112 of SEQ ID NO:2, and/or the beta chain variable domain comprising an amino acid sequence that has at least 80% identity to the sequence of amino acid residues 1-112 of SEQ ID NO:3;
optionally wherein the heterologous TCR comprises an:
a. αCDR1 having the sequence DRGSQA, SEQ ID NO:35,
b. αCDR2 having the sequence AVNSDSSYALNF, SEQ ID NO:36,
c. αCDR2 having the sequence AVNSDSGVALNF, SEQ ID NO:37,
d. αCDR1 having the sequence DRGSQA, SEQ ID NO:35 and αCDR2 having the sequence AVNSDSGVALNF, SEQ ID NO:37,
e. αCDR2 having the sequence AVNSQSGYALNF, SEQ ID NO: 38,
f. αCDR2 having the sequence AVNSQSGYSLNF, SEQ ID NO: 39,
g. αCDR2 having the sequence AVNSQSSYALNF, SEQ ID NO: 43
h. αCDR1 having the sequence DRGSQA, SEQ ID NO:35 and αCDR2 having the sequence AVNSQSGYALNF, SEQ ID NO: 38,
i. αCDR2 having the sequence AVNSQSGVALNF, SEQ ID NO: 39,
j. αCDR2 having the sequence AVNSQNGYALNF, SEQ ID NO: 40,
k. αCDR1 having the sequence DRGSFS, SEQ ID NO: 41,
l. αCDR1 having the sequence DRGSYS, SEQ ID NO: 42,
m. αCDR1 having the sequence DRGSYS, SEQ ID NO: 42 and αCDR2 having the sequence AVNSDSSYALNF SEQ ID NO: 36,
n. αCDR1 having the sequence DRGSYS, SEQ ID NO: 42 and αCDR2 having the sequence AVNSDSSYALNF SEQ ID NO: 36, or
o. αCDR1 having the sequence DRGSYS, SEQ ID NO: 42 and αCDR2 having the sequence AVNSQSGYALNF, SEQ ID NO: 38.

9-10. (canceled)

11. The method according to claim 1, wherein the population of modified immunoresponsive cells expressing or presenting a heterologous TCR further expresses or presents:
(a) a heterologous co-receptor, optionally wherein the co-receptor is a CD8 co-receptor; and/or
(b) a heterologous co-stimulatory ligand, optionally 4-1BBL or CD80.

12. The method according to claim 11, wherein:
(i) the heterologous CD8 co-receptor is heterodimer or homodimer, a CD8αb heterodimer or a CD8αα homodimer; and/or
(ii) the heterologous CD8 co-receptor comprises;
(a) a CDR 1 of at least 80% sequence identity to amino acid sequence VLLSNPTSG, SEQ ID NO:44, CDR 2 of at least 80% sequence identity to amino acid sequence YLSQNKPK SEQ ID NO:45 and CDR 3 of at least 80% sequence identity amino acid sequence LSNSIM SEQ ID NO:46,
(b) a CDR 1 of amino acid sequence VLLSNPTSG, SEQ ID NO:44, CDR 2 of amino acid sequence YLSQNKPK SEQ ID NO:45 and CDR 3 of amino acid sequence LSNSIM SEQ ID NO:46,
(c) an amino acid sequence having at least 80% sequence identity to amino acids number 22 to 235 of SEQ ID NO: 47, or 22 to 135 of SEQ ID NO: 47, or
(d) an amino acid sequence having 100% sequence identity to amino acids number 22 to 235 of sequence of SEQ ID NO: 47, or 22 to 135 of SEQ ID NO: 47.

13-14. (canceled)

15. The method of claim 1, wherein:
(i) the modified immunoresponsive cells are (a) B cells, T cells or natural killer (NK) cells, or (b) T cells, optionally a CD4⁺ T cells or CD8⁺ T cells; and/or
(ii) wherein the modified immunoresponsive cells is a population of CD4+ T cells; or CD8+ T cells, or a mixed population of CD4+ T cells and CD8+ T cells.

16. (canceled)

17. The method of claim 1, wherein:
(a) the modified immunoresponsive cells is administered continuously or intermittently; or
(b) the modified immunoresponsive cells is administered as multiple doses or is administered as a single dose.

18. (canceled)

19. The method of claim 17, wherein:
(i) the single or multiple doses are administered in one or more dosing cycles, optionally wherein the dose may be a fixed dose or a variable dose;
(ii) the modified immunoresponsive cells are administered at a dose of between about 500 million to about 1 billion cells, about 2 billion to about 5 billion cells or about 6 billion to about 10 billion cells; and/or
(iii) the modified immunoresponsive cells are administered as
(a) a single dose in each of one or more dosing cycles,
(b) one or more doses in each of one or more dosing cycles,
(c) a single dose on the first day of each of one or more dosing cycles,
(d) one or more doses in each of one or more dosing cycles, at least one dose being on the first day of each cycle, or
(f) a single dose.

20-21. (canceled)

22. The method according to claim 19, wherein:
(1) the dosing cycle is between 2 and 6 months or on disease progression;
(2) the dosing cycle is on:
(a) disease progression following a previous administration of modified immunoresponsive cells, and 12 weeks or more following the previous administration of modified immunoresponsive cells, and wherein
(b) the tumour and/or cancer expresses AFP and/or the subject serum AFP is greater than or equal to 100 ng/mL; or
(3) the dosing cycle is on:
(a) complete or partial response following a previous administration of modified immunoresponsive cells, or
(b) stable disease for a period of greater than or equal to 4 months; followed by disease progression following the previous administration of modified immunoresponsive cells, and greater than or equal to 12 weeks following the previous administration of modified immunoresponsive cells, and wherein
(c) the tumour and/or cancer expresses AFP and/or the subject serum AFP is greater than or equal to 100 ng/mL.

23-24. (canceled)

25. The method according to claim 1, wherein the modified immunoresponsive cells are administered intravenously or by intravenous infusion.

26. The method according to claim 1, wherein:
(a) prior to treatment the tumour and/or cancer cell AFP expression of the subject is greater than or equal to an intensity of 1+ in greater than or equal to 20% of tumour and/or cancer cells as determined by immunohistochemistry and non-cancerous AFP expression is less than or equal to 5% of cells for non-cancerous or non-tumour tissue at any intensity by immunohistochemistry,
(b) prior to treatment the serum level AFP of the subject is greater than or equal to 100 ng/mL and AFP expression is less than or equal to 5% of cells for non-cancerous or non-tumour tissue at any intensity by immunohistochemistry; and/or
(c) prior to treatment the subject has an Eastern Cooperative Oncology Group (ECOG) of 0 to 1 and/or Child-Pugh score of any one of 1, 2, 3, 4, 5 or 6 and/or measurable disease according to Response Evaluation Criteria in Solid Tumors (RECIST) 1.1.

27-28. (canceled)

29. The method according to claim 1, wherein if prior to treatment the subject has any one or more of:
(a) serum AFP levels within the normal range, less than 100 ng/mL or less than or equal to ng/mL,
(b) liver transplant,
(c) immunotherapy with PD-1 or PD-L1 antagonist ligands and/or cytotoxic chemotherapy,
(d) HLA-C*04:04 positive or HLA-B*51:03 positive status, or
(e) loco-regional therapy; then
the subject is excluded from the treatment.

30. The method according to claim 1, wherein:
(a) the subject is intolerant to a standard of care treatment, optionally wherein the standard of care treatment is selected from any one of Selected from Sorafenib, a PD1 or PD-L1 antagonist or inhibitor, Regorafenib, Cabozantinib, Sunitinib, Brivanib, Everolimus, Tivantinib, Linifanib;
(b) the cancer and/or tumour has been previously unsuccessfully treated with a standard of care treatment, optionally wherein the standard of care treatment is selected from any one of Sorafenib, a PD1 or PD-L1 antagonist or inhibitor, Regorafenib, Cabozantinib, Sunitinib, Brivanib, Everolimus, Tivantinib, Linifanib;
(c) the cancer and/or tumour has been previously unsuccessfully treated with locoregional therapy optionally selected from chemical and/or thermal percutaneous ablation and intraarterial chemoembolotherapy; and/or
(d) the subject has or wherein the cancer and/or tumour is; primary cancer, secondary cancer, relapsed cancer or refractory cancer or recurrent cancer or locally recurrent cancer or metastatic cancer, non-resectable cancer or locally confined, cancer with no surgical or radiotherapy option or inoperable cancer optionally wherein the cancer is not amenable to transplant or loco-regional therapy.

31-33. (canceled)

34. The method according to claim 1, wherein the cancer and/or tumour is liver cancer, or is liver cancer selected from; cholangiocarcinoma, liver angiosarcoma, hepatoblastoma, hepatocellular carcinoma (HCC), optionally wherein:
(a) the cancer is not amenable to transplant or resection;
(b) the cancer and/or tumour is hepatocellular carcinoma (HCC); and/or
(c) the liver cancer is coincident with any one or more of; diabetes, obesity, hepatitis B, hepatitis C, cirrhosis.

35-36. (canceled)

37. The method according to claim 1, wherein prior to administration of the modified immunoresponsive cells expressing or presenting a heterologous T-cell receptor (TCR) the subject undergoes lymphodepleting chemotherapy, optionally wherein:
(a) the lymphodepleting chemotherapy comprises administration of cyclophosphamide and fludarabine optionally at a dose of 500 mg/m²/d×3d cyclophosphamide and 20 mg/m2/d×3d fludarabine or at a dose of 600 mg/m²/d×3d cyclophosphamide and 30 mg/m2/d×4d; and/or
(b) the lymphodepleting chemotherapy is administered 7 to 5 or 7 to 4 days prior to administration of the modified immunoresponsive cells expressing or presenting a heterologous T-cell receptor (TCR).

38-39. (canceled)

40. The method according to claim 1, wherein:
(a) the subject has not received prior treatment for cancer and/or tumour or
(b) the subject has received prior cancer and/or tumour treatment and/or has failed to respond to prior cancer and/or tumour treatment.

41. (canceled)

42. The method according to claim 40, wherein the subject has received prior cancer and/or tumour treatment and/or has failed to respond to prior cancer and/or tumour treatment, further wherein:
(a) the prior treatment comprises; systemic and/or local therapy, optionally any one or more of surgery, radiation therapy cryotherapy, laser therapy, topical therapy and/or systemic therapy, for example any one or more of chemotherapy, hormonal therapy, targeted drugs, targeted chemotherapy, or immunotherapy;
(b) the prior treatment comprises a PD-L1 binding antagonist or PD-1 binding antagonist, optionally wherein the PD-1 axis binding antagonist or PD-L1 binding antagonist is an antibody;
(c) wherein the prior treatment comprises an Epidermal Growth Factor Receptor Antagonist, optionally Cetuximab;
(d) the prior treatment comprises chemotherapy comprising a platinum compound, optionally selected from Lipoplatin, Cisplatin, Carboplatin, Oxaliplatin, Nedaplatin, Triplatin tetranitrate, Phenanthriplatin, Satraplatin, Picoplatin;
(e) the prior treatment comprises chemotherapy comprising a chemotherapeutic agent selected from, methotrexate, capecitabine, taxane, anthracycline, paclitaxel, docetaxel, paclitaxel protein bound particles, doxorubicine, epirubicine, 5-fluorouracil, cyclophosphamide, afatinib, vincristine, etoposide or combinations thereof;
(f) the prior treatment comprises chemotherapy comprising a chemotherapeutic agent selected from, FEC: 5-fluorouracil, epirubicine, cyclophosphamide; FAC: 5-fluorouracil, doxorubicine, cyclophosphamide; AC: doxorubicine, cyclophosphamide; EC: epirubicine, cyclophosphamide; or
(g) the prior treatment comprises any one of Sorafenib, a PD1 or PD-L1 antagonist or inhibitor, Regorafenib, Cabozantinib, Sunitinib, Brivanib, Everolimus, Tivantinib, Linifanib, or locoregional therapy optionally selected from chemical and/or thermal percutaneous ablation and intraarterial chemoembolotherapy.

43-48. (canceled)

49. The method according to claim 42, wherein:
(a) the subject has not received prior treatment in recurrence less than or equal to 12 months since the last treatment or less than or equal to 6 months since the last treatment; or
(b) the subject has not received any prior adjuvant therapy (e.g. surgery followed by radiation and/or chemotherapy) or locoregional therapy in recurrence less than or equal to 12 months since the last treatment or in recurrence less than or equal to 6 months since the last treatment.

50. (canceled)

51. The method according to claim 1, wherein the treatment effectively extends or improves:
(a) the progression free survival,
(b) the time to progression,
(c) the duration of response,
(d) the overall survival,
(e) the objective response or objective response rate,
(f) the overall response or overall response rate,
(g) partial response or partial response rate,
(h) complete response or complete response rate;
(i) stable disease rate or median stable disease
(j) median progression free survival,
(k) median time to progression,
(l) median duration of response,
(m) median overall survival
(n) median objective response or median objective response rate,
(o) median overall response or median overall response rate,
(p) median partial response or median partial response rate,
(q) median complete response or median complete response, or
(r) median stable disease rate or median stable disease,
in comparison to placebo treatment or in comparison to prior to treatment or in comparison to without treatment or in comparison to treatment comprising a standard of care, optionally wherein the standard of care treatment is selected from any one of Sorafenib, a PD1 or PD-L1 antagonist or inhibitor, Regorafenib, Cabozantinib, Sunitinib Brivanib, Everolimus, Tivantinib, and Linifanib.


SEQUENCE LISTING (51 sequences)

SEQ ID NO: 1 - protein, 9 aa, Homo Sapiens
FMNKFIYEI

SEQ ID NO: 2 - protein, 205 aa, Artificial Sequence
  recombinant
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSDSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 3 - protein, 242 aa, Artificial Sequence
  recombinant
DSGVTQTPKHLITATGQRVTLRCSPRSGDLSVYWYQQSLDQGLQFLIQYYNGEERAKGNILERFSAQQFPDLHSELNLSSLELGDSALYFCASSLGGESEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 4 - protein, 205 aa, Artificial Sequence
  recombinant
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSDSGYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 5 - protein, 242 aa, Artificial Sequence
  recombinant
DSGVTQTPKHLITATGQRVTLRCSPRSGDLSVYWYQQSLDQGLQFLIQYYNGEERAKGNILERFSAQQFPDLHSELNLSSLELGDSALYFCASSLGGESEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 6 - protein, 205 aa, Artificial Sequence
  recombinant
QKEVEQNSGPLSVPEGAIASLNCTYSDRGSQAFFWYRQYSGKSPELIMSIYSNGDKEDGRFTAQLNKASQYVSLLIRDSQPSDSATYLCAVNSDSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 7 - protein, 205 aa, Artificial Sequence
  recombinant
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSDSSYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 8 - protein, 205 aa, Artificial Sequence
  recombinant
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSDSGVALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 9 - protein, 205 aa, Artificial Sequence
  recombinant
QKEVEQNSGPLSVPEGAIASLNCTYSDRGSQAFFWYRQYSGKSPELIMSIYSNGDKEDGRFTAQLNKASQYVSLLIRDSQPSDSATYLCAVNSDSGVALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 10 - protein, 205 aa, Artificial Sequence
  recombinant
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSQSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 11 - protein, 205 aa, Artificial Sequence
  recombinant
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSQSGYSLNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 12 - protein, 205 aa, Artificial Sequence
  recombinant
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSQSSYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 13 - protein, 205 aa, Artificial Sequence
  recombinant
QKEVEQNSGPLSVPEGAIASLNCTYSDRGSQAFFWYRQYSGKSPELIMSIYSNGDKEDGRFTAQLNKASQYVSLLIRDSQPSDSATYLCAVNSQSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 14 - protein, 205 aa, Artificial Sequence
  recombinant
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSQSGVALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 15 - protein, 205 aa, Artificial Sequence
  recombinant
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSQNGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 16 - protein, 205 aa, Artificial Sequence
  recombinant
QKEVEQNSGPLSVPEGAIASLNCTYSDRGSFSFFWYRQYSGKSPELIMSIYSNGDKEDGRFTAQLNKASQYVSLLIRDSQPSDSATYLCAVNSDSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 17 - protein, 205 aa, Artificial Sequence
  recombinant
QKEVEQNSGPLSVPEGAIASLNCTYSDRGSYSFFWYRQYSGKSPELIMSIYSNGDKEDGRFTAQLNKASQYVSLLIRDSQPSDSATYLCAVNSDSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 18 - protein, 205 aa, Artificial Sequence
  recombinant
QKEVEQNSGPLSVPEGAIASLNCTYSDRGSYSFFWYRQYSGKSPELIMSIYSNGDKEDGRFTAQLNKASQYVSLLIRDSQPSDSATYLCAVNSDSSYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 19 - protein, 205 aa, Artificial Sequence
  recombinant
QKEVEQNSGPLSVPEGAIASLNCTYSDRGSYSFFWYRQYSGKSPELIMSIYSNGDKEDGRFTAQLNKASQYVSLLIRDSQPSDSATYLCAVNSDSGVALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 20 - protein, 205 aa, Artificial Sequence
  recombinant
QKEVEQNSGPLSVPEGAIASLNCTYSDRGSYSFFWYRQYSGKSPELIMSIYSNGDKEDGRFTAQLNKASQYVSLLIRDSQPSDSATYLCAVNSQSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 21 - protein, 615 aa, Artificial Sequence
  recombinant
CAAAAAGAAGTTGAGCAGAATTCTGGACCCCTCAGTGTTCCAGAGGGAGCCATTGCCTCTCTCAACTGCACTTACAGTGACCGAGGTTCCCAGTCCTTCTTCTGGTACAGACAATATTCTGGGAAAAGCCCTGAGTTGATAATGTCCATATACTCCAATGGTGACAAAGAAGATGGAAGGTTTACAGCACAGCTCAATAAAGCCAGCCAGTATGTTTCTCTGCTCATCAGAGACTCCCAGCCCAGTGATTCAGCCACCTACCTCTGTGCCGTGAATAGTGATTCCGGGTATGCACTCAACTTCGGCAAAGGCACCTCGCTGTTGGTCACACCCCATATCCAGAACCCTGACCCTGCCGTGTACCAGCTGAGAGACTCTAAGTCGAGTGACAAGTCTGTCTGCCTATTCACCGATTTTGATTCTCAAACAAATGTGTCACAAAGTAAGGATTCTGATGTGTATATCACAGACAAATGTGTGCTAGACATGAGGTCTATGGACTTCAAGAGCAACAGTGCTGTGGCCTGGAGCAACAAATCTGACTTTGCATGTGCAAACGCCTTCAACAACAGCATTATTCCAGAAGACACCTTCTTCCCCAGCCCAGAAAGTTCC

SEQ ID NO: 22 - DNA, 726 bp, Artificial Sequence
  recombinant
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcctcgggggggaatctgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagac

SEQ ID NO: 23 - DNA, 1867 bp, Artificial Sequence
  recombinant
gctagccgccaccatgatgaagtccctgcgggtgctgctggtcatcctgtggctgcagctgtcctgggtctggtcccagcagaaagaggtggagcagaacagcggccctctgagcgtgcccgagggcgctatcgccagcctgaactgcacctacagcgacagaggcagccagagcttcttctggtacagacagtacagcggcaagagccccgagctgatcatgagcatctacagcaacggcgacaaagaggacggccggttcaccgcccagctgaacaaggccagccagtacgtgtccctgctgatccgggacagccagcccagcgacagcgccacctacctgtgcgccgtgaacagcgactccggctacgccctgaacttcggcaagggcaccagcctgctggtgacaccccacattcagaaccccgaccccgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgacttcgcctgcgccaacgccttcaacaacagcatcatccccgaggacacatttttcccaagccccgagagcagctgcgacgtcaaactggtggagaagtccttcgagacagacaccaacctgaacttccagaacctgagcgtgatcggcttcagaatcctgctgctgaaggtggccggcttcaatctgctgatgaccctgcggctgtggtccagcggcagcagagccaagagaagcggatccggcgccaccaacttcagcctgctgaagcaggccggcgacgtggaggaaaaccctggccctaggatgggcttccggctgctgtgctgcgtggccttctgcctgctgggagccggccctgtggatagcggcgtgacccagacccccaagcacctgatcaccgccaccggccagagagtgaccctgcgctgcagccctagaagcggcgacctgtccgtgtactggtatcagcagagcctggaccagggactgcagttcctcatccagtactacaacggcgaggaacgggccaagggcaacatcctggaaagattcagcgcccagcagttccccgacctgcacagcgagctgaacctgagcagcctggaactgggcgactccgccctgtacttctgcgccagcagcctgggcggcgagagcgaacagtacttcggccctggcacccggctgacggtaaccgaggacctgaagaacgtgttccccccagaggtggccgtgttcgagccctctgaggccgagatcagccacacccagaaagccaccctggtctgcctggccaccggcttctaccccgaccacgtggaactgtcttggtgggtgaacggcaaagaggtgcacagcggcgtcagcaccgaccctcagcccctgaaagagcagcccgccctgaacgacagccggtactgcctgagcagcagactgcgggtgtccgccaccttctggcagaacccccggaaccacttcagatgccaggtgcagttctacggcctgagcgagaacgacgagtggacccaggaccgggccaagcctgtgacccagatcgtgtctgccgaagcatgggggcgcgccgattgcggcttcacaagcgagagctaccagcagggcgtgctgagcgccaccatcctgtacgagatcctgctgggcaaggccaccctgtacgccgtgctggtgtccgctctggtgctgatggccatggtgaaacggaaggacagccggggctaataagtcgac

SEQ ID NO: 24 - protein, 614 aa, Artificial Sequence [sequence omitted]

SEQ ID NO: 25 - DNA, 45 bp, Artificial Sequence
  recombinant
gaattccatatgcaaaaagaagttgaacaaaattctggacccctc

SEQ ID NO: 26 - DNA, 36 bp, Artificial Sequence
  recombinant
ttgtcagtcgacttagagtctctcagctggtacacg

SEQ ID NO: 27 - DNA, 45 bp, Artificial Sequence
  recombinant
gaattccatatggattctggagttacacaaaccccaaagcacctg

SEQ ID NO: 28 - DNA, 32 bp, Artificial Sequence
  recombinant
tagaaaccggtggccaggcacaccagtgtggc

SEQ ID NO: 29 - protein, 6 aa, Artificial Sequence
  recombinant
DRGSQS

SEQ ID NO: 30 - protein, 6 aa, Artificial Sequence
  recombinant
IYSNGD

SEQ ID NO: 31 - protein, 12 aa, Artificial Sequence
  recombinant
AVNSDSGYALNF

SEQ ID NO: 32 - protein, 5 aa, Artificial Sequence
  recombinant
SGDLS

SEQ ID NO: 33 - protein, 6 aa, Artificial Sequence
  recombinant
YYNGEE

SEQ ID NO: 34 - protein, 11 aa, Artificial Sequence
  recombinant
ASSLGGESEQY

SEQ ID NO: 35 - protein, 6 aa, Artificial Sequence
  recombinant
DRGSQA

SEQ ID NO: 36 - protein, 12 aa, Artificial Sequence
  recombinant
AVNSDSSYALNF

SEQ ID NO: 37 - protein, 12 aa, Artificial Sequence
  recombinant
AVNSDSGVALNF

SEQ ID NO: 38 - protein, 12 aa, Artificial Sequence
  recombinant
AVNSQSGYALNF

SEQ ID NO: 39 - protein, 12 aa, Artificial Sequence
  recombinant
AVNSQSGYSLNF

SEQ ID NO: 40 - protein, 12 aa, Artificial Sequence
  recombinant
AVNSQNGYALNF

SEQ ID NO: 41 - protein, 6 aa, Artificial Sequence
  recombinant
DRGSFS

SEQ ID NO: 42 - protein, 6 aa, Artificial Sequence
  recombinant
DRGSYS

SEQ ID NO: 43 - protein, 12 aa, Artificial Sequence
  recombinant
AVNSQSSYALNF

SEQ ID NO: 44 - protein, 9 aa, Homo sapiens
VLLSNPTSG

SEQ ID NO: 45 - protein, 9 aa, Homo sapiens
VLLSNPTSG

SEQ ID NO: 46 - protein, 6 aa, Homo sapiens
LSNSIM

SEQ ID NO: 47 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 48 - DNA, 745 bp, Homo sapiens
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtcggttcaagagctaaaagaagtggtagtggtgcccctgtga

SEQ ID NO: 49 - protein, 205 aa, Artificial Sequence; identical to SEQ ID NO: 13
  recombinant

SEQ ID NO: 50 - protein, 242 aa, Artificial Sequence; identical to SEQ ID NO: 3
  recombinant

SEQ ID NO: 51 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV